(19) United States
(12) Patent Application Publication
(10) Pub. No.: US 2021/0292752 A1
(43) Pub. Date: Sep. 23, 2021

(54) Method for Isolating or Identifying Cell, and Cell Mass

(71) Applicants: Spiber Inc., Yamagata (JP); The University of Tokyo, Tokyo (JP)

(72) Inventors: Kana Ishida, Tsuruoka-shi, Yamagata (JP); Soh Ishiguro, Bunkyo-ku, Tokyo (JP); Nozomu Yachie, Bunkyo-ku, Tokyo (JP); Chikako Sato, Tsuruoka-shi, Yamagata (JP); Junichi Sugahara, Tsuruoka-shi, Yamagata (JP)

(73) Assignees: Spiber Inc., Yamagata (JP); The University of Tokyo, Tokyo (JP)

(21) Appl. No.: 17/266,566

(22) PCT Filed: Aug. 13, 2019
(86) PCT No.: PCT/JP2019/031872

§ 371 Date: Feb. 5, 2021

(30) Foreign Application Priority Data
Aug. 13, 2018 (JP) 2018-152403
Jan. 28, 2019 (JP) 2019-012268

(51) Int. Cl. C12N 15/10

(52) U.S. Cl. CPC C12N 15/1086; C12N 15/1065

(57) ABSTRACT

Disclosed is a method for isolating or identifying target clone cells from a cell population, the method including steps of: preparing a cell population into which a barcode sequence and at least one reporter protein abnormal expression cassette linked to the barcode sequence are introduced; introducing a barcode sequence recognition module targeting an arbitrary barcode sequence and a nucleic acid mutation repair enzyme into cells; repairing a nucleic acid mutation causing abnormal expression occurring in the at least one reporter protein abnormal expression cassette by expression of a complex of the barcode sequence recognition module and the nucleic acid mutation repair enzyme in a cell containing the target barcode sequence, to induce normal expression of the reporter protein; and isolating or identifying target clone cells in which the reporter protein is expressed.

### TECHNICAL FIELD

The present invention relates to a method for isolating or identifying cells and a cell population.

### BACKGROUND ART

It has been pointed out that heterogeneity of a cell population is important for cell differentiation, and growth of cancer cells or ontogeny. For example, it is revealed by genome analysis that cultured cell systems that cause malignancy or differentiation of cancer cells or serve as a model thereof are heterogeneous and different cell clones, which is regarded as one of the causes that make cancer treatment difficult. On the other hand, in the study related to a heterogeneous cell population, it is shown that “a cell clone that will exhibit a particular trait in the future” is buried in the heterogeneous cell population in a highly complex initial state, and thus, the cell clone cannot be identified, isolated, and cultured from various cells.

Since it is difficult to reveal the mechanism that causes the malignancy of the cancer with only the genome analysis, it is required to separate and analyze the heterogeneous cell populations by a certain method. In a cell separation method according to the related art, such as flow cytometry, cells are usually selected based on a surface marker of a cell. Therefore, the cell separation method is a method useful for selecting immune cells for which surface antigens are identified and the like. However, a gene set which enables selective separation of a target clone from a population is required to sort and analyze cells by a conventional method using a surface antigen marker or the like. Therefore, it is difficult to sort and analyze cells whose expression of a marker is not clear or a population that cannot be separated by a known marker. For example, it has been pointed out that an unknown sub-population exists in a process in which hematopoietic stem cells are differentiated into blood cells and matured, but at present, these cell populations cannot be sorted and analyzed. In addition, for example, in a process of inducing iPS cells from fibroblasts, a phenomenon in which the induction efficiency differs for each clone has been found, but at present, it is difficult to sort clones with a high induction efficiency and to analyze gene expression, a DNA methylation state, and the like.

Further, cells repeatedly interact with each other in a population, resulting in a change in intracellular kinetics in each cell. A process of acquiring drug resistance in cancer cells may be given as an example. It is urgently required for development of ideal anti-cancer drugs to identify the response of a cancer cell population to anti-cancer drugs. On the other hand, it has not been clarified how molecular dynamics such as a genomic structure or gene expression of each cancer cell clone acts on and responds to the entire cancer cell population due to the difficulty in its analysis with today's technology. For example, the team of Novartis in the USA and Harvard University conducted the experiment in which highly complex DNA barcodes are introduced into genomes of non-small cell lung cancer-derived cell lines using lentivirus to measure a change in cell proliferation under exposure to anti-cancer drugs (Non Patent Literature 1). A method for simultaneously tracking an increase and decrease of cell clones different from each other is established by diminishing the diversity of the DNA barcodes in the population under long-term exposure to a plurality of anti-cancer drugs. However, it is not possible to analyze, even by the method, how the molecular dynamics of cell clones in which amplification of specific genes or a change in cell morphology is observed varies under a cell population environment over time

### CITATION LIST

### Non Patent Literature

Non Patent Literature 1: Bhang H E et al., Nat Med., 2015, 21(5): 440-8

### SUMMARY OF INVENTION

### Technical Problem

An object of the present invention is to provide a method for isolating or identifying arbitrary cells from a cell population and a cell population used in the method.

### Solution to Problem

The present inventors found a method capable of identifying and isolating arbitrary cell clones from a cell population by using a bar code technology for simultaneous labeling of cell populations and a nucleic acid editing technology, thereby completing the present invention.

The present invention provides, for example, the following inventions.

[1]

A method for isolating or identifying target clone cells from a cell population, the method including:

a step (i) of preparing a cell population into which a barcode sequence and at least one reporter protein abnormal expression cassette linked to the barcode sequence are introduced;

a step (ii) of introducing a barcode sequence recognition module targeting an arbitrary barcode sequence and a nucleic acid mutation repair enzyme into cells;

a step (iii) of repairing a nucleic acid mutation causing abnormal expression occurring in the at least one reporter protein abnormal expression cassette by expression of a complex of the barcode sequence recognition module and the nucleic acid mutation repair enzyme in a cell containing the target barcode sequence, to induce normal expression of the reporter protein; and a step (iv) of isolating or identifying target clone cells in which the reporter protein is expressed.

[2]

The method according to [1], wherein the complex converts one or more nucleotides into another one or more nucleotides or deletes the one or more nucleotides, or inserts one or more nucleotides, at a site of the nucleic acid mutation.

[3]

The method according to [1] or [2], wherein the nucleic acid mutation is a mutation in a sequence (ATG) encoding methionine which first appears from an N-terminus.

[4]

The method according to [3], wherein the ATG is not included in the barcode sequence.

[5]

The method according to any one of [1] to [4], wherein the barcode sequence recognition module is a guide RNA,

the nucleic acid mutation repair enzyme is linked to a Cas protein, and

the guide RNA contains a sequence complementary to at least a part of the barcode sequence.

[6]

A cell population in which a barcode sequence and at least one reporter protein abnormal expression cassette linked to the barcode sequence are introduced into individual cells.

[7]

The cell population according to [6], wherein a nucleic acid mutation in the at least one reporter protein abnormal expression cassette is a mutation in a sequence (ATG) encoding methionine which first appears from an N-terminus.

[8]

The cell population according to [6] or [7], wherein the ATG is not included in the barcode sequence.

[9]

The cell population according to any one of [6] to [8], wherein the cell population includes a complex in which a nucleic acid sequence recognition module targeting an arbitrary barcode and a nucleic acid mutation repair enzyme are bound to each other.

### Advantageous Effects of Invention

According to the present invention, it is possible to provide a method for isolating or identifying arbitrary cells from a cell population and a cell population used in the method.

### BRIEF DESCRIPTION OF DRAWINGS

FIG. 1 illustrates fluorescence micrographs showing results of Example 1.

FIG. 2 illustrates graphs showing fluorescence intensities of RFPs in Example 1, in which targe indicates a case where a targe sgRNA is used, and scrambled indicates a case where a scrambled sgRNA is used.

FIG. 3 is a schematic diagram illustrating an experiment of Example 2.

FIG. 4 illustrates graphs showing results of Example 2, in which % described in each graph indicates a percentage of a population in which GFP fluorescence is observed.

FIG. 5 is a graph showing an ATG conversion efficiency in a case where each barcode is used in Example 3.

FIG. 6 illustrates graphs showing results obtained by using different combinations of inducers and cell lines in each system in Example 4.

FIG. 7 illustrates graphs showing a relationship between a percentage of GFP-positive cells (activation %) and a false positive (error %) in each system in Example 4.

FIG. 8 illustrates examples of a colony in which RFP expression is predicted in Example 5, in which the left shows a result obtained in a case where an sgRNA (sgRNA_BC7) is used, and the right shows a result obtained in a case where an sgRNA (sgRNA_BC8) is used.

FIG. 9 illustrates results of determining sequences near barcode sequences in sampled colonies by a next-generation sequencer in Example 5, in which a shaded area indicates the barcode sequence, and an enclosure line indicates a start codon ATG repaired by a mutation.

### DESCRIPTION OF EMBODIMENTS

Hereinafter, embodiments of the present invention will be described in detail. However, the present invention is not limited to the following embodiments.

A method for isolating or identifying target clone cells from a cell population according to an embodiment includes the following steps (i) to (iv) of:

(i) preparing a cell population into which a barcode sequence and at least one reporter protein abnormal expression cassette linked to the barcode sequence are introduced;

(ii) introducing a barcode sequence recognition module targeting an arbitrary barcode sequence and a nucleic acid mutation repair enzyme into cells;

(iii) repairing a nucleic acid mutation causing abnormal expression occurring in the at least one reporter protein abnormal expression cassette by expression of a complex of the barcode sequence recognition module and the nucleic acid mutation repair enzyme in a cell containing the target barcode sequence, to induce normal expression of the reporter protein; and

(iv) isolating or identifying target clone cells in which the reporter protein is expressed.

In the present invention, the cells are not particularly limited, and for example, various cells such as cancer cells, hematopoietic stem cells, blood cells, fibroblasts, and iPS cells can be used.

The cell population refers to a group of cells. The cell population may include homogeneous cells in which only a single clone is present, but when the cell population is a heterogeneous cell population, the effects of the present invention are significantly exhibited, which is preferable. The heterogeneous cell population refers to a group of cells in which a plurality of clones is present.

In the present invention, the target clone cells are isolated or identified by selection based on expression of the reporter protein. The target clone cell is a cell to be isolated or identified, and may be a single cell or a group of progeny cells in which the above cells are proliferated.

[Step (i)]

The step (i) is a step of preparing a cell population into which a barcode sequence and at least one reporter protein abnormal expression cassette (genetic circuit) linked to the barcode sequence are introduced.

The barcode sequence of the present invention is a sequence such as a tag (JP H10-507357 A or JP 2002-518060 A), a zip code (JP 2001-519648 A) or an orthonormalization sequence (JP 2002-181813 A), or a barcode sequence (Xu, Q., Schlabach, M. R., Hannon, G. J. et al. (2009) PNAS 106, 2289-2294). The barcode sequence may be a barcode sequence using a DNA sequence (DNA barcode sequence), or may be a barcode sequence using a peptide nucleic acid (PNA) which is an analog of a DNA or RNA. It is desirable that the barcode sequence has a small cross-reactivity (cross-hybridization). In addition, the barcode sequence may be 8 to 30 bases in length, 10 to 25 bases in length, 15 to 20 bases in length, 17 to 20 bases in length, or 16 to 18 bases in length. In addition, it is preferable that the barcode does not contain a sequence (ATG) corresponding to a start codon, and it is more preferable that the barcode does not contain both a sequence corresponding to a start codon and a sequence corresponding to a termination codon (TAA, TAG, or TGA), from the viewpoint of stability of protein expression of a gene located downstream. A specific example of the barcode includes a DNA barcode composed of a total of 17 bases having four consecutive units and one base N (((WSNS)₄N), in which four bases WSNS (W=A/T, S=G/C, N=A/T/G/C) form one unit. Since, in theory, each WSNS unit of the barcode does not contain a sequence corresponding to a start codon and a sequence corresponding to a termination codon, it is expected to prevent initiation and termination of translation of a gene (for example, a reporter gene) located downstream in an unintended reading frame, and thus, it is expected to contribute to stability and high sensitivity of the method according to the present embodiment.

The reporter protein abnormal expression cassette is designed so that a reporter protein is not normally expressed due to a nucleic acid mutation in a reporter protein expression cassette. In a case where the reporter protein is normally expressed, a target selection can be performed based on the expression thereof. Abnormal expression of the reporter protein may include not only a case where the reporter protein is not expressed at all due to the presence of the nucleic acid mutation, but also a case where the target selection cannot be performed based on the expression of the reporter protein due to an abnormal structure of the expressed protein or an excessively low expression level of the protein. However, the cause of the abnormal expression of the reporter protein is not limited to the nucleic acid mutation in the gene encoding the reporter protein, and may also be a nucleic acid mutation in a promoter for expressing a reporter protein or the like. The reporter protein abnormal expression cassette is designed so that the reporter protein is normally expressed in a case where the nucleic acid mutation is corrected.

The nucleic acid mutation that causes the abnormal expression is a mutation of a nucleotide in the reporter protein abnormal expression cassette, and is preferably a mutation of a base of a nucleotide in a polynucleotide encoding a reporter protein. The number of mutations of the base of the nucleotide is not particularly limited, and the mutation may occur in one to five bases, one to four bases, one to three bases, one or two bases, or one base. In addition, the mutations of the bases may be continuous, or a plurality of mutations may separately occur. A type of the mutation may be any of substitution, insertion, deletion, and a combination thereof. The mutation is preferably a mutation in ATG (methionine corresponding to the start codon) which first appears from an N-terminus in an amino acid sequence of the reporter protein, and more preferably a mutation in which A of the ATG is substituted with G.

The reporter protein expression cassette is not particularly limited as long as it is a polynucleotide capable of expressing a reporter protein in a cell. A typical example of the expression cassette includes a polynucleotide containing a promoter and a reporter protein coding sequence arranged to be under control of the promoter.

The promoter is not particularly limited, and examples thereof include constitutive promoters such as a CMV promoter, an EF1a promoter, a UbiC promoter, a PGK promoter, a U6 promoter, and a CAG promoter. As the promoter of the reporter protein expression cassette, a CMV promoter is preferably used.

The reporter protein is not particularly limited, and examples thereof include a light-emitting (chromogenic) protein that emits light (develops color) by reacting with a specific substrate, and a fluorescent protein that emits fluorescence by excitation light. Examples of the light-emitting (chromogenic) protein include luciferase, 13-galactosidase, chloramphenicol acetyltransferase, and β-glucuronidase. Examples of the fluorescent protein include GFP, Azami-Green, ZsGreen, GFP2, EGFP, HyPer, Sirius, BFP, CFP, Turquoise, Cyan, TFP1, YFP, Venus, ZsYellow, Banana, KusabiraOrange, RFP, DsRed, AsRed, Strawberry, Jred, KillerRed, Cherry, HcRed, and mPlum. Examples of a drug resistance reporter protein include proteins encoded by drug resistance genes such as a chloramphenicol resistance gene, a tetracycline resistance gene, a neomycin resistance gene, an erythromycin resistance gene, a spectinomycin resistance gene, a kanamycin resistance gene, a hygromycin resistance gene, and a puromycin resistance gene. The reporter protein also includes a fusion protein with a light-emitting (chromogenic) protein or a fluorescent protein, and a protein obtained by adding a known protein tag, a known signal sequence, or the like to a light-emitting (chromogenic) protein or a fluorescent protein. In addition, the reporter protein may be a part of a known protein as long as it is normally expressed.

The reporter protein coding sequence is not particularly limited as long as it is a base sequence encoding an amino acid sequence of the reporter protein. As described above, since the reporter protein may be a part of a known protein, the reporter protein coding sequence may be a base sequence partially encoding an ORF of a known protein. For example, methionine which appears in the middle of an amino acid sequence of a known protein can be used as a start codon.

The reporter protein abnormal expression cassette is linked to each barcode sequence. The reporter protein abnormal expression cassette and each barcode sequence may be directly linked or indirectly linked, or each barcode sequence may be incorporated into the reporter protein abnormal expression cassette. In a case where the barcode sequence is incorporated into the reporter protein abnormal expression cassette, a sequence encoding a reporter protein containing a mutation may be arranged directly downstream of the barcode sequence, or some other nucleic acids may be arranged between the barcode sequence and a sequence encoding a reporter protein containing a mutation. A distance from the 3′ end of the barcode sequence to the nucleic acid mutation in the reporter protein abnormal expression cassette (in a case where the barcode sequence is arranged upstream), or a distance from the nucleic acid mutation in the reporter protein abnormal expression cassette to the 5′ end of the barcode sequence (in a case where the barcode sequence is arranged downstream) may be, for example, 0 to 3 bases in length, 0 to 2 bases in length, or 0 to 1 base in length, in terms of the number of bases.

A method for introducing the barcode sequence and the at least one reporter protein abnormal expression cassette linked to the barcode sequence into a cell is not particularly limited, and a method known to those skilled art, for example, a method using an expression vector, can be used.

The expression vector can be produced, for example, by linking the corresponding DNA to the downstream of the promoter in a suitable expression vector. In addition, the expression vector can optionally contain a selectable marker such as a terminator, a repressor, a drug resistance gene, or an auxotrophic complementary gene, and a replication origin that can function in a host.

The expression vector can be introduced according to a known method (for example, a lysozyme method, a competent method, a PEG method, a CaCl₂ coprecipitation method, an electroporation method, a microinjection method, a particle gun method, a lipofection method, and an Agrobacterium method), depending on a type of the host.

[Step (ii)]

The step (ii) is a step of introducing a barcode sequence recognition module targeting an arbitrary barcode sequence and a nucleic acid mutation repair enzyme into cells.

The arbitrary barcode sequence refers to a barcode sequence selected from a group of the barcode sequences described above.

The barcode sequence recognition module is a module targeting the selected barcode sequence, and has a barcode recognition region. The barcode recognition region is preferably a sequence complementary to at least a part of the barcode sequence.

As the barcode sequence recognition module of the present invention, for example, it is possible to use a module using a CRISPR-Cas system, a module using a CRISPR-Cas system in which at least one DNA cleavage ability of Cas is inactivated (hereinafter, referred to as “CRISPR-mutant Cas”, CRISPR-mutant Cpf1 is also included), or a fragment which contains a DNA-binding domain of a protein that can specifically bind to a DNA, such as a restriction enzyme, a transcription factor, or an RNA polymerase, in addition to a zinc finger motif, a TAL effector, and a PPR motif, and does not have a DNA double-strand cleavage ability, but the present invention is not limited thereto. Preferred examples thereof include CRISPR-mutant Cas, a zinc finger motif, a TAL effector, and a PPR motif.

The zinc finger motif is obtained by linking 3 to 6 Cys2His2 zinc finger units different from each other (one finger recognizes about 3 bases), and the zinc finger motif can recognize a target nucleotide sequence of 9 to 18 bases. The zinc finger motif can be produced by a known method such as a modular assembly method (Nat Biotechnol (2002) 20: 135-141), an OPEN method (Mol Cell (2008) 31: 294-301), a CoDA method (Nat Methods (2011) 8: 67-69), and an Escherichia coli one-hybrid method (Nat Biotechnol (2008) 26: 695-701). Patent Literature 1 can be referred to as for details of the production of the zinc finger motif.

The TAL effector has a repeating structure of modules with about 34 amino acids as a unit, and binding stability and base specificity thereof are determined by the 12^{th} and 13^{th} amino acid residues (called RVD) of one module. Since each of the modules is highly independent, a TAL effector specific to a target nucleotide sequence can be produced by simply connecting the modules. A production method using open resources (a REAL method (Curr Protoc Mol Biol (2012) Chapter 12: Unit 12.15), a FLASH method (Nat Biotechnol (2012) 30: 460-465), a Golden Gate method (Nucleic Acids Res (2011) 39: e82), or the like) is established for the TAL effector, and a TAL effector for a target nucleotide sequence can be relatively simply designed. Patent Literature 2 can be referred to as for details of the production of the TAL effector.

The PPR motif is configured to recognize a specific nucleotide sequence by repeats of PPR motifs consisting of 35 amino acids and recognizing one nucleic acid base, and the PPR motif recognizes a target base only by the 1^{st}, 4^{th}, and ii (−2)^{nd} amino acids of each motif. Motif repetition has no dependency, and is free of interference of motifs on both sides. Therefore, similarly to the TAL effector, a PPR protein specific to the target nucleotide sequence can be produced by simply connecting PPR motifs. JP 2013-128413 A can be referred to as for details of the production of the PPR motif.

In addition, in a case where a fragment of a restriction enzyme, a transcription factor, an RNA polymerase, or the like is used, since the DNA binding domains of these proteins are well known, a fragment containing the domain and having no DNA double-strand cleavage ability can be easily designed and constructed.

In a case where the CRISPR-Cas system is used, a target double-stranded DNA sequence is recognized by a guide RNA containing a sequence complementary to a target barcode sequence, so that an arbitrary sequence can be targeted simply by synthesizing an oligo DNA capable of specifically forming a hybrid with the target barcode sequence.

In a more preferred embodiment of the present invention, the CRISPR-Cas system is preferably used, and a CRISPR-Cas system (CRISPR-mutant Cas) in which a Cas protein (for example, nickase) whose at least one DNA cleavage ability is inactivated is used is more preferably used.

An example of the barcode sequence recognition module in a case where the CRISPR-Cas system is used includes a guide RNA.

For example, the barcode sequence recognition module may be a guide RNA consisting of a CRISPR-RNA (crRNA) containing a sequence complementary to the target barcode sequence (barcode sequence recognition region) and a trans-activating RNA (tracrRNA) required for recruiting of a Cas protein (chimeric RNA).

A guide RNA coding sequence is not particularly limited as long as it is a base sequence encoding the guide RNA.

The guide RNA is not particularly limited as long as it is used in a CRISPR/Cas system, and for example, it is possible to use various guide RNAs capable of inducing a Cas protein to a target site by binding to the target site and the Cas protein.

In the present specification, the target site to which the guide RNA binds is a site consisting of a Proto-spacer Adjacent Motif (PAM) sequence, a barcode sequence adjacent to the 5′ side of the PAM sequence (target strand), and a strand complementary to the barcode sequence (non-target strand). A distance from the sequence on the most 5′ side of the PAM sequence to the nucleic acid mutation in the reporter protein abnormal expression cassette may be, for example, 15 to 20 bases in length in terms of the number of bases.

The PAM sequence varies depending on a type of a Cas protein to be used. For example, a PAM sequence corresponding to a Cas9 protein (type II) derived from S. pyogenes is 5′-NGG, a PAM sequence corresponding to a Cas9 protein (type I-A1) derived from S. solfataricus is 5′-CCN, a PAM sequence corresponding to a Cas9 protein (type I-A2) derived from S. solfataricus is 5′-TCN, a PAM sequence corresponding to a Cas9 protein (type I-B) derived from H. walsbyl is 5′-TTC, a PAM sequence corresponding to a Cas9 protein (type I-E) derived from E. coli is 5′-AWG, a PAM sequence corresponding to a Cas9 protein (type I-F) derived from E. coli is 5′-CC, a PAM sequence corresponding to a Cas9 protein (type I-F) derived from P. aeruginosa is 5′-CC, a PAM sequence corresponding to a Cas9 protein (type II-A) derived from S. Thermophilus is 5′-NNAGAA, a PAM sequence corresponding to a Cas9 protein (type II-A) derived from S. agalactiae is 5′-NGG, a PAM sequence corresponding to a Cas9 protein derived from S. aureus is 5′-NGRRT or 5′-NGRRN, a PAM sequence corresponding to a Cas9 protein derived from N. meningitidis is 5′-NNNNGATT, and a PAM sequence corresponding to a Cas9 protein derived from T. denticola is 5′-NAAAAC.

The guide RNA contains a sequence (may be referred to as a CRISPR RNA (crRNA) sequence) involved in binding to a target site, and the guide RNA can bind to a target site by complementarily (preferably, complementarily and specifically) binding the crRNA sequence to a sequence excluding a sequence complementary to the PAM sequence of a non-target strand. In the present embodiment, the crRNA sequence complementarily binds to the barcode sequence.

Specifically, among the crRNA sequences, a sequence that binds to the barcode sequence has, for example, 80% or more or 90% or more, preferably 95% or more, more preferably 98% or more, still more preferably 99% or more, and particularly preferably 100% identity with the barcode sequence. It is known that, 12 bases on the 3′ side of the sequence binding to the target sequence, among the crRNA sequences, are important for the binding of the guide RNA to the target site. Therefore, in a case where, among the crRNA sequences, a sequence that binds to the barcode sequence is not completely the same as the barcode sequence, it is preferable that a base different from the barcode sequence is a base other than the 12 bases of the 3′ side of the sequence binding to the barcode sequence, among the crRNA sequences.

The tracrRNA sequence is not particularly limited. The tracrRNA sequence typically is an RNA composed of a sequence of about 50 to 100 bases in length capable of forming a plurality of (generally, three) stem loops, and the sequence varies depending on a type of the Cas protein to be used. As the tracrRNA sequence, it is possible to employ various known sequences depending to the type of the Cas protein to be used.

In general, the guide RNA contains the crRNA sequence and tracrRNA sequence described above. According to an aspect, the guide RNA may be a single-stranded RNA (sgRNA) containing a crRNA sequence and a tracrRNA sequence, or may be an RNA complex formed by complementarily binding an RNA containing a crRNA sequence to an RNA containing a tracrRNA sequence.

For example, in a case where the guide RNA is a single-stranded RNA (sgRNA) containing a crRNA sequence and a tracrRNA sequence, specific examples of an expression cassette of the guide RNA include a polynucleotide containing a promoter, a site for inserting a crRNA coding sequence arranged to be under control of the promoter, and a tracrRNA coding sequence arranged downstream of the site, and a polynucleotide containing a promoter and an sgRNA coding sequence arranged to be under control of the promoter. As another example, in a case where the guide RNA is an RNA complex formed by complementarily binding an RNA containing a crRNA sequence to an RNA containing a tracrRNA, a typical example of an expression cassette of the guide RNA includes a combination of an expression cassette containing a promoter and a “crRNA sequence-containing RNA” coding sequence arranged to be under control of the promoter (or a site for inserting a crRNA coding sequence) (a crRNA expression cassette), and an expression cassette containing a promoter and a “tracrRNA sequence-containing RNA” coding sequence arranged to be under control of the promoter (tracrRNA expression cassette).

The site for inserting the crRNA coding sequence is not particularly limited as long as it has a sequence suitable for insertion of a polynucleotide containing an arbitrary crRNA coding sequence. An example of the site includes a sequence containing one restriction enzyme site or a plurality of restriction enzyme sites.

The nucleic acid mutation repair enzyme is not particularly limited as long as it is an enzyme capable of repairing a nucleic acid mutation causing an abnormality occurring in the reporter protein abnormal expression cassette, but it is preferable that the complex thereof with a barcode sequence recognition module described below converts one or more nucleotides into another one or more nucleotides or deletes the one or more nucleotides, or inserts one or more nucleotides, at a site of the nucleic acid mutation. Examples of the nucleic acid mutation repair enzyme include nucleic acid base converting enzymes such as cytidine deaminase, adenosine deaminase, and guanosine deaminase. The origin of the nucleic acid mutation repair enzyme is not particularly limited, and for example, in the case of cytidine deaminase, Petromyzon marinus cytidine deaminase 1 (PmCDA1) derived from a lamprey, or an activation-induced cytidine deaminase (AID) derived from a vertebrate (for example, a mammal such as a human, a pig, a cow, a dog, or a chimpanzee, a bird such as a chicken, an amphibian such as a xenopus, a fish such as a zebrafish, a sweetfish, or a channel catfish, and the like) (AICDA) can be used.

In a case where the CRISPR-Cas system is used, the nucleic acid mutation repair enzyme may be directly or indirectly linked to the Cas protein.

A Cas protein coding sequence is not particularly limited as long as it is a base sequence encoding an amino acid sequence of the Cas protein.

The Cas protein is not particularly limited as long as it is used in the CRISPR/Cas system, and for example, it is possible to use various proteins capable of binding to a target site in a state of forming a complex with a guide RNA and cleaving the target site. Proteins derived from various organisms are known as the Cas protein, and examples of the Cas protein include a Cas9 protein (type II) derived from S. pyogenes, a Cas9 protein (type I-A1) derived from S. solfataricus, a Cas9 protein (type I-A2) derived from S. solfataricus, a Cas9 protein (type I-B) derived from H. walsbyl, a Cas9 protein (type I-E) derived from E. coli, a Cas9 protein (type I-F) derived from E. coli, a Cas9 protein (type I-F) derived from P. aeruginosa, a Cas9 protein (type II-A) derived from S. Thermophilus, a Cas9 protein (type II-A) derived from S. agalactiae, a Cas9 protein derived from S. aureus, a Cas9 protein derived from N. meningitidis, a Cas9 protein derived from T. denticola, and a Cpf1 protein (type V) derived from F. novicida. Among them, a Cas9 protein is preferred, and a Cas9 protein endogenously present in bacteria belonging to the genus Streptococcus is more preferred.

The Cas protein may be a wild-type double-strand cleavage Cas protein or a nickase-type Cas protein. In general, the double-strand cleavage Cas protein has a domain involved in cleavage of a target strand (RuvC domain) and a domain involved in cleavage of a non-target strand (HNH domain). An example of the nickase-type Cas protein includes a protein having a mutation that impairs its cleavage activity (for example, its cleavage activity is reduced to ½, ⅕, 1/10, 1/100, 1/1,000 or less) in any one of the two domains of the double-strand cleavage Cas protein. Any of a Cas protein whose ability to cleave both strands of a double-stranded DNA is inactivated and a Cas protein whose ability to cleave one strand alone is inactivated and having nickase activity can be used. As such a mutation, in the case of Cas9 derived from Streptococcus pyogenes (SpCas9), nCas and dCas can be used. In the present specification, nCas refers to a D10A mutant in which the 10^{th} Asp residue is converted into an Ala residue and lacking an ability to cleave a strand opposite to the strand forming a complementary strand to a guide RNA, or a H840A mutant in which the 840^{th} His residue is converted into an Ala residue and lacking an ability to cleave a strand complementary to a guide RNA, and dCas refers to a double mutant thereof. Similarly, mutant Cas other than nCas and dCas can be used.

The Cas protein may have a mutation (for example, substitution, deletion, insertion, addition, or the like) in an amino acid sequence as long as its activity is not impaired. From this viewpoint, the Cas protein may be a wild-type double-strand cleavage Cas protein, or a protein consisting of an amino acid sequence having, for example, 85% or more, preferably 90% or more, more preferably 95% or more, and still more preferably 98% or more identity with an amino acid sequence of a nickase-type Cas protein based on the wild-type double-strand cleavage Cas protein and having activity thereof (activity to bind to a target site in a state of forming a complex with a guide RNA to cleave the target site). Alternatively, from the same viewpoint, the Cas protein may be a wild-type double-strand cleavage Cas protein, or a protein consisting of an amino acid sequence obtained by substitution, deletion, addition, or insertion (preferably, conservative substitution) of one amino acid or a plurality of amino acids (for example, 2 to 100, preferably 2 to 50, more preferably 2 to 20, still more preferably 2 to 10, even still more preferably 2 to 5, and particularly preferably 2 amino acids) in an amino acid sequence of a nickase-type Cas protein based on the wild-type double-strand cleavage Cas protein and having activity thereof (activity to bind to a target site in a state of forming a complex with a guide RNA to cleave the target site). As an inactive Cas9 mutant, for example, nCas and dCas described above can be used.

A known protein tag, a signal sequence, and a protein such as an enzyme protein may be added to the Cas protein. Examples of the protein tag include biotin, a His tag, a FLAG tag, a Halo tag, an MBP tag, an HA tag, a Myc tag, a V5 tag, and a PA tag. An example of the signal sequence includes a nuclear localization signal. Examples of the enzyme protein include various histone-modifying enzymes and a deaminase.

As a genome editing technology using CRISPR, in addition to a case of using CRISPR-Cas9, a case using CRISPR-Cpf1 has been reported (Zetsche B., et al., Cell, 163: 759-771 (2015)). Examples of Cpf1 capable of genome editing in mammal cells include Cpf1 derived from Acidaminococcus sp. BV3L6 and Cpf1 derived from Lachnospiraceae bacterium ND2006, but are not limited thereto. In addition, examples of a mutant Cpf1 lacking a DNA cleavage ability include a D917A mutant in which the 917^{th} Asp residue of Cpf1 derived from Francisella novicida U112 (FnCpf1) is converted into an Ala residue, an E1006A mutant in which the 1,006^{th} Glu residue is converted into an Ala residue, and a D1255A mutant in which the 1,255^{th} Asp residue is converted into an Ala residue, but any mutant Cpf1 lacking a DNA cleavage ability can be used in the present invention without limitation to these mutants.

In a case where a CRISPR-Cas system is used, it is preferable that the barcode sequence recognition module is a guide RNA, the nucleic acid mutation repair enzyme binds to a Cas protein, and the guide RNA contains a sequence complementary to at least a part of the barcode sequence. With such a configuration, the method for isolating or identifying the target clone cells can be implemented with higher specificity (less false positives) and higher expression efficiency.

In the present embodiment, the complex of the barcode sequence recognition module and the nucleic acid mutation repair enzyme and the barcode sequence are in contact with each other by introducing the complex or a nucleic acid encoding the complex into a cell containing a desired barcode sequence. Therefore, the barcode sequence recognition module and the nucleic acid mutation repair enzyme may form a complex before being introduced into a cell, or may form a complex in a cell after being introduced into the cell. Considering introduction and an expression efficiency, it is desirable to introduce a nucleic acid-modifying enzyme complex into a cell in a form of a nucleic acid encoding the complex rather than the nucleic acid-modifying enzyme complex itself so as to express the complex in the cell.

Therefore, it is preferable that the barcode sequence recognition module and the nucleic acid mutation repair enzyme (furthermore, in some cases, an inhibitor of base excision repair described below) are prepared as nucleic acids encoding fusion proteins thereof, or as nucleic acids encoding them, respectively, in a form capable of forming a complex in a host cell after translation into a protein using a binding domain, an intein, and the like. Here, the nucleic acid may be a DNA or an RNA. The DNA is preferably a double-stranded DNA. The DNA is provided in a form of an expression vector placed under control of a functional promoter in a host cell. The RNA is preferably a single-stranded RNA.

The cell into which a nucleic acid encoding the nucleic acid-modifying enzyme complex is introduced can include cells of all species ranging from cells of bacteria such as E. coli, which is a prokaryote, or microorganisms such as yeast, which is a lower eukaryote to cells of higher eukaryotes such as vertebrates including a mammal such as a human, insects, and plants.

As a method for introducing the complex into the cell, a method known to those skilled in the art, such as a method using, for example, an expression vector can be used as in the step (i).

An expression vector containing a DNA encoding a nucleic acid sequence recognition module and/or a nucleic acid base converting enzyme or an inhibitor of base excision repair can be prepared, for example, by linking the DNA to the downstream of the promoter in an appropriate expression vector.

The promoter may be any promoter suitable for a host used for expression of a gene. In a conventional method using DSB, since the viability of the host cell may be significantly reduced due to toxicity, it is desirable that the number of cells is increased by the start of induction using an inducible promoter. However, since sufficient cell proliferation can also be obtained by expressing the nucleic acid-modifying enzyme complex of the present invention, a constitutive promoter can be used without limitation.

The expression vector can optionally contain a selectable marker such as a terminator, a repressor, a drug resistance gene, or an auxotrophic complementary gene, and a replication origin that can function in a host.

An RNA encoding a nucleic acid sequence recognition module and/or a nucleic acid base converting enzyme or an inhibitor of base excision repair can be prepared by, for example, transcription into an mRNA in an in vitro transcription system known per se by using a vector encoding a DNA encoding the above-described nucleic acid sequence recognition module and/or the nucleic acid base converting enzyme as a template.

The expression vector can be introduced according to a known method (for example, a lysozyme method, a competent method, a PEG method, a CaCl₂) coprecipitation method, an electroporation method, a microinjection method, a particle gun method, a lipofection method, and an Agrobacterium method), depending on a type of the host.

[Step (iii)]

The step (iii) is a step of repairing a nucleic acid mutation causing abnormal expression occurring in the at least one reporter protein abnormal expression cassette by expression of a complex of the barcode sequence recognition module and the nucleic acid mutation repair enzyme in a cell containing the target barcode sequence, to induce normal expression of the reporter protein.

When the complex of the barcode sequence recognition module and the nucleic acid mutation repair enzyme is expressed in a cell, the barcode sequence recognition module specifically recognizes and binds to a target barcode sequence in a target double-stranded DNA, and the nucleic acid mutation causing the abnormal expression is repaired by an action of the nucleic acid mutation repair enzyme linked to the barcode sequence recognition module. For example, in a case where the nucleic acid mutation repair enzyme is a nucleic acid base converting enzyme, a base conversion occurs in a sense strand or an antisense strand of a nucleic acid mutation site (the entire or a part of the nucleic acid mutation or their vicinity) due to the action of the nucleic acid base converting enzyme linked to the barcode sequence recognition module, resulting in mismatch in the double-stranded DNA. When the mismatch is not correctly repaired, and when a base of an opposite strand is repaired to pair with a base of a converted strand, or another nucleotide is further substituted or one to several tens of bases are deleted or inserted during repair, various mutations are introduced. A specific example of using a CRISPR/Cas system using a reporter protein abnormal expression cassette in which A of a start codon ATG of a reporter protein is converted into G will be described below. When a complex of a guide RNA and cytidine deaminase is expressed, the guide RNA recognizes a target barcode sequence, so that a double strand is broken by an action of Cas9, and thus, the cytidine deaminase acts thereon, whereby cytosine is converted into uracil. The generated mismatch sequence is converted into the corresponding sequence by a repair mechanism, and a single base conversion of C→U (T) is thus achieved. Therefore, the mutation to G in ATG, which is the cause of the abnormal expression is repaired to A (correction to a wild-type), and the reporter protein can be normally expressed.

The nucleic acid mutation introduced for repair by the nucleic acid mutation repair enzyme may be degraded by a base excision repair (BER) mechanism carried out by glycosylase or the like. Therefore, it is preferable to inhibit such a base excision repair mechanism. BER can be inhibited by introducing the above-described inhibitor of BER or a nucleic acid encoding the same, or by introducing a low molecular weight compound inhibiting BER. Alternatively, BER in the cells can be inhibited by suppressing expression of a gene involved in a BER pathway. The expression of the gene can be suppressed, for example, by introducing into a cell an expression vector capable of expressing a siRNA, an antisense nucleic acid, or polynucleotides thereof capable of specifically suppressing expression of a gene involved in the BER pathway. In addition, the expression of the gene can be suppressed by knocking out a gene involved in the BER pathway.

An example of a method for inhibiting BER includes introducing of an inhibitor of BER or a nucleic acid encoding the same together with a barcode sequence recognition module and a nucleic acid mutation repair enzyme into a cell in the step (ii). The inhibitor of base excision repair is not particularly limited as long as it effectively inhibits BER, but it is preferably an inhibitor of a DNA glycosylase located upstream of the BER pathway, from the viewpoint of an efficiency. Examples of the inhibitor of a DNA glycosylase include an inhibitor of a thymine-DNA glycosylase, an inhibitor of a uracil-DNA glycosylase, an inhibitor of an oxoguanine-DNA glycosylase, and an inhibitor of an alkylguanine-DNA glycosylase. For example, in a case where cytidine deaminase (for example, PmCDA1) is used as the nucleic acid base converting enzyme, it is preferable that an inhibitor of a uracil-DNA glycosylase is used in order to inhibit the repair of U:G or G:U mismatch in the DNA caused by the mutation.

Examples of the inhibitor of a uracil-DNA glycosylase include a uracil-DNA glycosylase inhibitor (Ugi) derived from PBS1, which is a Bacillus subtilis bacteriophage, or a uracil-DNA glycosylase inhibitor (Ugi) derived from PBS2, which is a Bacillus subtilis bacteriophage (Wang, Z., and Mosbaugh, D. W. (1988) J. Bacteriol. 170, 1082-1091), but are not limited thereto. Any inhibitor can be used in the present invention as long as it is the DNA mismatch repair inhibitor. In particular, it is more preferable to use Ugi derived from PBS2 because it is known that Ugi derived from PBS2 has an effect of suppressing mutation or cleavage and recombination other than C to T on the DNA.

As described above, in the base excision repair (BER) mechanism, when the base is removed by the DNA glycosylase, an AP endonuclease nicks a non-basic site (AP site), and then, the AP site is completely removed by an exonuclease. When the AP site is removed, a DNA polymerase creates a new base using the base of the opposite strand as a template, and finally, the nick is filled with a DNA ligase, thereby completing the repair. It is known that, a mutant AP endonuclease whose enzyme activity is lost, but ability to bind to the AP site is retained competitively inhibits BER. Therefore, these mutant AP endonucleases can also be used as the inhibitor of base excision repair of the present invention. The origin of the mutant AP endonuclease is not particularly limited, and for example, an AP endonuclease derived from E. coli, yeast, a mammal (for example, a human, a mouse, a pig, a cow, a horse, a monkey, or the like), or the like can be used. An example of the mutant AP endonuclease whose enzyme activity is lost, but ability to bind to the AP site is retained includes a protein in which an active site or a binding site for Mg, which is a cofactor, is mutated. In a case of human Ape1, examples thereof include E96Q, Y171A, Y171F, Y171H, D210N, D210A, and N212A.

In a case where the barcode sequence recognition module forms a complex with the nucleic acid mutation repair enzyme before being introduced into a cell as described above, it is possible to provide the barcode sequence recognition module as a fusion protein with the nucleic acid mutation repair enzyme and/or the inhibitor of base excision repair, or it is possible to fuse a protein binding domain such as an SH3 domain, a PDZ domain, a GK domain, or a GB domain, and a binding partner thereof with each of the barcode sequence recognition module and the nucleic acid base converting enzyme and/or the inhibitor of base excision repair, whereby to provide the barcode sequence recognition module as a protein complex through the interaction between the domain and the binding partner thereof. Alternatively, an intein can be fused with each of the nucleic acid sequence recognition module and the nucleic acid mutation repair enzyme and/or the inhibitor of base excision repair so as to link them by ligation after synthesis of each protein.

[Step (iv)]

The step (iv) is a step of isolating or identifying target clone cells in which the reporter protein is expressed.

The method for isolating or identifying target clone cells is not particularly limited, and a method known to those skilled in the art can be appropriately used based on the type of reporter protein and the like. For example, in a case where the reporter protein is a fluorescent protein, cell clones are isolated from selected pools by sorting cells using a flow cytometer, and in a case where the reporter protein is a drug resistance gene, cell clones are isolated based on expression of a marker gene by drug administration, and cell clones are isolated by forming single colonies by seeding cells at a low density. Here, the isolated target clone cell may be a single cell without needing to be a cell group.

In a cell population according to the present embodiment, a barcode sequence and at least one reporter protein abnormal expression cassette linked to the barcode sequence are introduced into individual cells. The barcode sequence and the at least one reporter protein abnormal expression cassette linked to the barcode sequence, the type of the cell, the method for introducing them into the cell, and the like are the same as described above.

A nucleic acid mutation in the at least one reporter protein abnormal expression cassette is preferably a mutation in a sequence (ATG) encoding methionine which first appears from an N-terminus. In addition, it is preferable that a sequence corresponding to a start codon is not contained in the barcode sequence. In addition, it is preferable that the cell population includes a complex in which a nucleic acid sequence recognition module targeting an arbitrary barcode and a nucleic acid mutation repair enzyme are bound to each other.

### EXAMPLES

### Plasmids Used in Examples

Some of plasmids used in the following Examples are shown in Table 1.

TABLE 1

| | | SEQ ID |
|---|---|---|
| Plasmid Name | Plasmid Map (Benchling) | NO |
| ADH1p-dCas9 | https://benchling.com/s/seq- | 1 |
| | LAfF2rjirEfeVJCofN | |
| ADH1p-dCas9-PmCDA1- | https://benchling.com/s/seq- | 2 |
| UGI | cz9Uy3GnmJmkQ6SnBJb9 | |
| backbone_ADH1p-filler- | https://benchling.com/s/seq- | 3 |
| ΔRFP [HIS3] | IilDkdYV2FfUudu1gph4 | |
| reporter_ADH1p-PAM-BC2- | https://benchling.com/s/seq- | 4 |
| 9th GTG-RFP [HIS3] | Rhif2NyAwqVf0oc4m0Pp | |
| reporter_ADH1p-PAM-BC2- | https://benchling.com/s/seq- | 5 |
| 9th ATG-RFP [HIS3] | ju1xyXxAQjjxaffJZXt7 | |
| backbone_SNR52p-filler- | https://henchling.com/s/seq- | 6 |
| sgRNA scaffold | 0SLTQNjct9j1nkdQcGw1 | |
| sgRNA_SNR52p-target | https://benchling.com/s/seq- | 7 |
| BC2 | 2ZnrSYsuXE9abFBhpqPI | |
| sgRNA_SNR52p-scrambled | https://benchling.com/s/seq- | 8 |
| | Rt9TJCWMbok6Q0E3gYm7 | |

All of the plasmids of Table 1 were designed based on the data registered in Benchling (produced by Benchling, Inc.).

### Example 1 Demonstration Experiment in Yeast Cells (1)

### <Reporter Expression and Abnormal Expression Vectors>

The following RFP vector was constructed as a reporter abnormal expression vector.

5′ ADH1 promoter-PAM-barcode-9^{th}GTG-RFP-ADH1 terminator 3′ (SEQ ID NO: 4)

The 9^{th}RFP refers to an RFP containing an ORF which is shorter than a normal ORF and is obtained by deleting a sequence using methionine that is the 9^{th} amino acid appearing in an amino acid sequence of an RFP as a start codon, the deleted sequence being arranged upstream (N-terminus side) of the start codon. The 9^{th}GTG-RFP refers to a mutant obtained by converting ATG which is the start codon and encodes methionine in the 9^{th}RFP into GTG. 5′ AGCGTGTCAGGGTGACC 3′ (SEQ ID NO: 9) from a random DNA barcode represented by (WSNS)₄N was used as the barcode sequence (barcode).

A reporter expression vector was constructed (also referred to as “9^{th}ATG-RFP”, SEQ ID NO: 5) in the same manner as that of the above reporter expression vector, except that methionine which was the start codon was not mutated in the 9^{th}RFP.

### <Cas9 Protein-Nucleic Acid Mutation Repair Enzyme Expression Vector (Target-AID)>

A vector consisting of 5′ADH1 promoter-Cas9 variant (-PmCDA1-UGI)-CYC1 terminator 3′ (SEQ ID NO: 2) was used as a Cas9 protein-nucleic acid mutation repair enzyme expression vector. 5′ADH1 promoter-dCas9-CYC1 terminator 3′ (SEQ ID NO: 1) was used as a negative control.

### <Barcode Sequence Recognition Module (Guide RNA) Expression Vector>

A barcode sequence recognition module (guide RNA) expression vector (Target sgRNA, SEQ ID NO: 7) was constructed as follows.

A vector consisting of 5′ SNR52 promoter-filler-sgRNA scaffold-SUP4 terminator 3′ (SEQ ID NO: 6) was used as a backbone. A filler sequence was removed from the backbone, and a spacer sequence corresponding to the barcode sequence (barcode recognition region, 5′ CACGGTCACCCTGACACGCT 3′ (SEQ ID NO: 10)) was inserted instead of the filler sequence.

A vector consisting of 5′ SNR52 promoter-CTGAAAAAGGAAGGAGTTGA-sgRNA scaffold-SUP4 terminator 3′ (Scrambled sgRNA, SEQ ID NO: 8) was used as a negative control that does not target a target sequence.

### <Transformation of Yeast>

A Y8800 strain for yeast two-hybrid was used as yeast. A commercially available kit (Frozen-EZ Yeast Transformation II™, ZYMO RESEARCH) was used to transform the above-described vector. SD-His-Leu-Ura+Ade was used as an agar medium, and colonies were obtained by culturing at 30° C. for about 48 hours to 72 hours after inoculation. Compositions of the selective agar media used in Examples are shown in Table 2.

TABLE 2

| | SD- | | SD- | | SD-His- | | SD-His-Leu- | |
|---|---|---|---|---|---|---|---|---|
| Selective Medium | His + Ade | | Leu + Ade | | Leu + Ade | | Ura + Ade | |
| Nitrogen Base For Yeast | 1.34 | g | 1.34 | g | 1.34 | g | 1.34 | g |
| (Containing Ammonium Sulfate) | | | | | | | | |
| DO Mix (-Ade/-Ura/-RHLW) | 0.4 | g | 0.4 | g | 0.4 | g | 0.4 | g |
| ddH₂O | 171 | mL | 171 | mL | 171 | mL | 171 | mL |
| Bacto Agar (Final | 6 | g | 6 | g | 6 | g | 6 | g |
| Concentration 3%) | | | | | | | | |
| Sum | 179 | mL | 179 | mL | 179 | mL | 179 | mL |
| 40% of Glucose | 10 | mL | 10 | mL | 10 | mL | 10 | mL |
| 12 mg/mL Adenine Solution | 3 | mL | 3 | mL | 3 | mL | 3 | mL |
| 100 mM Arginine Solution | 1.6 | mL | 1.6 | mL | 1.6 | mL | 1.6 | mL |
| 100 mM Histidine Solution | — | mL | 1.6 | mL | — | mL | — | mL |
| 40 mM Tryptophan Solution | 1.6 | mL | 1.6 | mL | 1.6 | mL | 1.6 | mL |
| 100 mM Leucine Solution | 1.6 | mL | — | mL | — | mL | — | mL |
| 20 mM Uracil Solution | 1.6 | mL | 1.6 | mL | 1.6 | mL | — | mL |
| ddH₂O | 1.6 | mL | 1.6 | mL | 3.2 | mL | 4.8 | mL |
| Total | 200 | mL | 200 | mL | 200 | mL | 200 | mL |

### <Observation of RFP Expression>

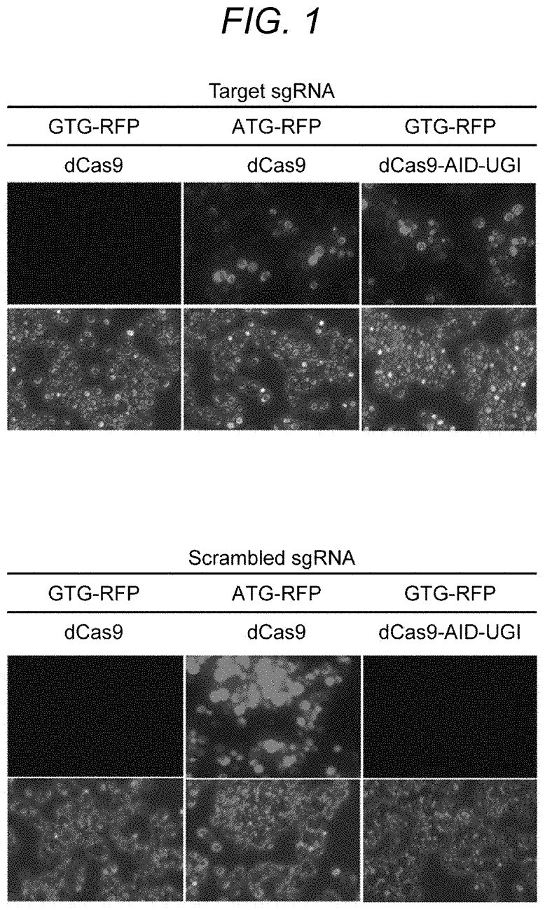

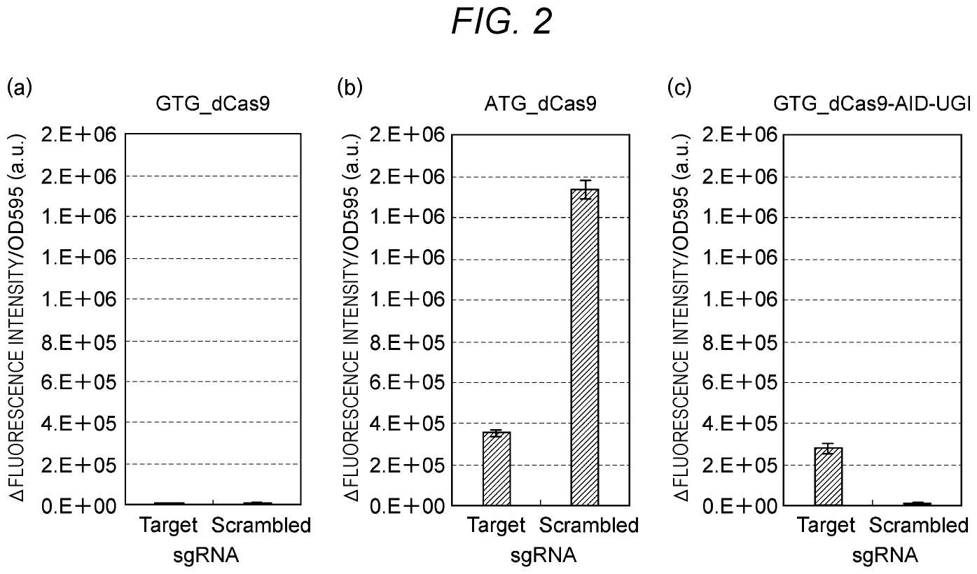

The yeast colonies were directly suspended in the selective liquid media shown in Table 3 or cultured therein for 5 hours or longer, a supernatant was removed, about 2 μL of a fungus body was placed on a slide glass and fixed with a cover glass, and then cells were observed using a fluorescence microscope (BZ-X710, KEYENCE Corporation). The results are illustrated in FIG. 1. In addition, the results obtained by measuring fluorescence intensity of the RFP using a microplate reader (Infinite F200 Pro-FL/T, TECAN Group Ltd.) are illustrated in FIG. 2. In a case where Target sgRNA and dCas9-AID-UGI were used, fluorescence of the RFP was partially observed. It was considered that this was resulted by the correction of the start codon by single base genome editing with PmCDA1, which was the nucleic acid mutation repair enzyme. In a case where a BY4741 strain was used as yeast, the same results were obtained. It was suggested that the above method may be useful as a reporter system for a cell isolation method.

TABLE 3

| | | Per 1 L | |
|---|---|---|---|
| | | (Aqueous | |
| Selective Medium | Manufacturer | Solution) | |
| Nitrogen Base for Yeast | MP Biomedicals | 1.52 | g |
| DO Mix (As for Composition, | — | 1.25 | g |
| Refer To Table 4) | | | |
| 5N Sodium Hydroxide | Wako Pure Chemical | 501 | μ1 |
| | Industries, Ltd. | | |
| Glucose | Wako Pure Chemical | 20 | g |
| | Industries, Ltd. | | |
| Adenine | Wako Pure Chemical | 180 | mg |
| | Industries, Ltd. | | |
| Arginine | Wako Pure Chemical | 139.4 | mg |
| | Industries, Ltd. | | |
| Tryptophan | Wako Pure Chemical | 65.3 | mg |
| | Industries, Ltd. | | |

TABLE 4
Do Mix (-Ade/-Ade/-Arg/-His/-Leu/-Trp/-Ura)

| | Weight | | Catalog |
|---|---|---|---|
| | (g) | Vendor | No. |
| L-alanine | 5 | PEPTIDE INSTITUTE, | 2701 |
| | | INC. | |
| L-asparaginemonohydrate | 5.68 | PEPTIDE INSTITUTE, | 2703 |
| | | INC. | |
| L-aspartic acid | 5 | PEPTIDE INSTITUTE, | 2704 |
| | | INC. | |
| L-cysteine | 5 | Wako Pure Chemical | 033-20655 |
| | | Industries, Ltd. | |
| L-glutamine | 5 | PEPTIDE INSTITUTE, | 2707 |
| | | INC. | |
| L-glutamic acid | 5 | Wako Pure Chemical | 072-00501 |
| | | Industries, Ltd. | |
| L-glycine | 5 | Wako Pure Chemical | 077-00735 |
| | | Industries, Ltd. | |
| Inositol | 5 | LKT Laboratories, | 15357 |
| | | Inc. | |
| L-isoleucine | 5 | PEPTIDE INSTITUTE, | 2712 |
| | | INC. | |
| L-lysine | 5 | Wako Pure Chemical | 124-06212 |
| | | Industries, Ltd. | |
| L-methionine | 5 | PEPTIDE INSTITUTE, | 2715 |
| | | INC. | |
| p-aminobenzoic acid | 5 | Wako Pure Chemical | 019-02335 |
| | | Industries, Ltd. | |
| L-phenylalanine | 5 | PEPTIDE INSTITUTE, | 2717 |
| | | INC. | |
| L-proline | 5 | PEPTIDE INSTITUTE, | 2718 |
| | | INC. | |
| L-serine | 5 | PEPTIDE INSTITUTE, | 2719 |
| | | INC. | |
| L-threonine | 5 | PEPTIDE INSTITUTE, | 2720 |
| | | INC. | |
| L-tyrosine | 5 | PEPTIDE INSTITUTE, | 2722 |
| | | INC. | |
| L-valine | 5 | PEPTIDE INSTITUTE, | 2723 |
| | | INC. | |

### Example 2 Demonstration Experiment in Human Cells

### <Reporter Abnormal Expression Vector>

Each mutant EGFP in which an arbitrary barcode sequence was added from a random DNA represented by (WSNS)₄N to a lentiviral vector pLVSIN-CMV-Puro (Takara) (a sequence was acquired from pLV-eGFP, and ATG encoding a start codon was converted into GTG) was amplified by a PCR method, and the amplified mutant EGFP was cloned.

### <Placement of Reporter in Cell Genome>

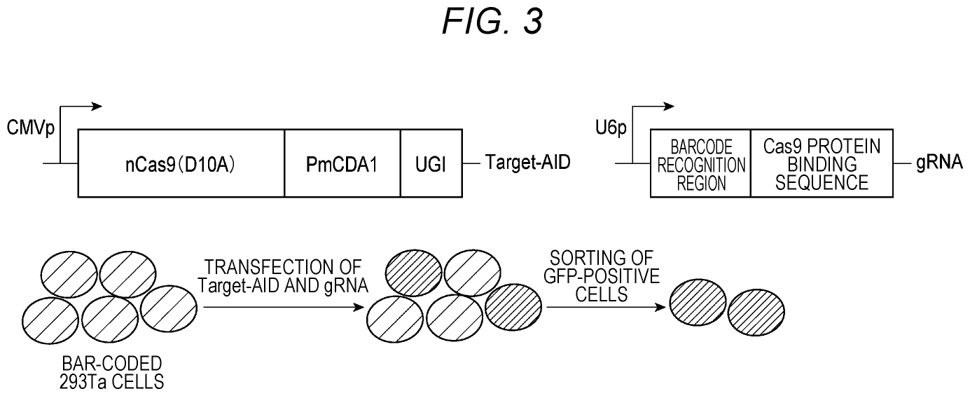

The reporter abnormal expression vector was transfected into HEK293Ta cells together with helper plasmids pMD2.G (https://www.addgene.org/12259/ (SEQ ID NO: 11) and psPAX2 (https://www.addgene.org/12260/ (SEQ ID NO: 12) to produce lentivirus. The lentivirus particles were collected, and then, the HEK293Ta cells were infected with the virus, thereby obtaining a cell line with genome into which the present reporter was incorporated by puromycin selection (barcoded 293Ta cells of FIG. 3).

### <Demonstration Experiment on Functionality of CloneSelect Reporter System>

Simultaneously, a guide RNA that targets a T002 barcode sequence (AACTATAACATCATTTCGTG, SEQ ID NO: 14) (On-target gRNA, SEQ ID NO: 15) (pLV-CS-076 (lentiGuide-T002)) and a negative control guide RNA that does not target the T002 barcode sequence (Off-target gRNA, SEQ ID NO: 16) (pLV-CS-077(lentiGuide-Scramble1)) were obtained from a random DNA barcode sequence group used in the construction of the reporter abnormal expression vector (pLV-CS-110(lenti-T002-GTG-EGFP), SEQ ID NO: 13). The Cas9 protein-nucleic acid mutation repair enzyme expression vector (Target-AID, CMVp-Sp nCas9-PmCDA1-UGI, SEQ ID NO: 17) (pcDNA3.1_pCMV-nCas-PmCDA1-ugi pH1-gRNA(HPRT)) and the guide RNA expression vector were transfected into the cell line, and after three days, a percentage of GFP-positive cells was analyzed by a flow cytometer FACS Verse (manufactured by BD Biosciences).

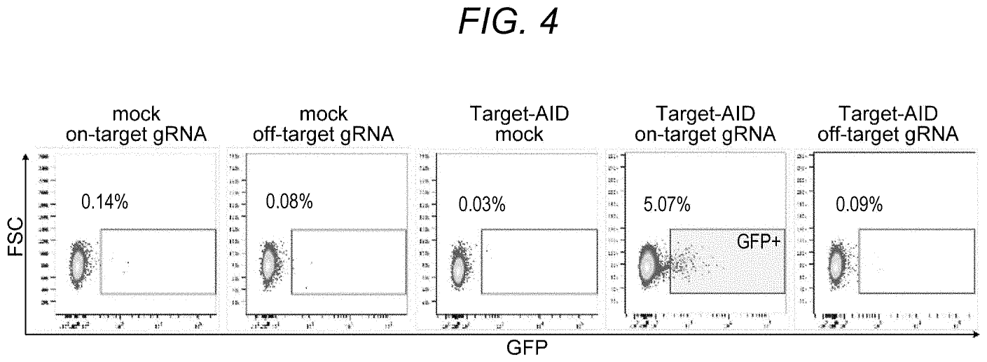

As a result, in a case where a Target-AID and an On-target gRNA were used, GFP fluorescence was confirmed in about 5% of the populations (FIG. 4). On the other hand, in a case where an off-target guide RNA was used, it was confirmed that a percentage of the GFP-positive cells was 0.09% or less, which was very low. Therefore, the detected GFP fluorescence was considered to be due to the correction of the start codon by single base genome editing. It was suggested that the above method may be useful as a reporter system for a cell isolation method.

### Example 3 Conversion Efficiency of Start Codon

In the method described in Example 2, the reporter plasmid was placed into the HEK293Ta cells by controlling the infection efficiency of the target cells with the lentivirus to 10% or less, and assuming that an average of one copy of the barcodes was incorporated into each genome. As a result, cultured human cells (HEK293Ta) having about 100 types of barcoded reporter GFP in the genome were prepared.

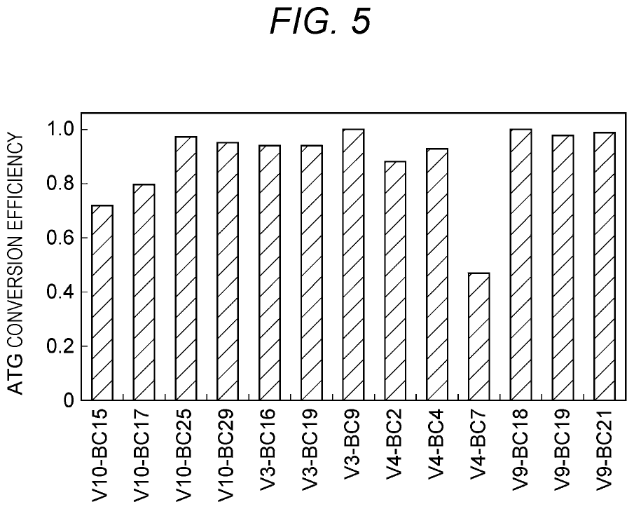

Each of the Cas9 protein-nucleic acid mutation repair enzyme expression vector (CMVp-Sp nCas9-PmCDA1-UGI) and the guide RNA expression vector targeting 13 types of barcodes (refer to FIG. 5) was transfected, and after three days, the GFP-positive cells were sorted using a flow cytometer FACS Jazz (manufactured by BD Biosciences).

TABLE 5

| | | | SEQ ID |
|---|---|---|---|
| | Name | Sequence | NO |
| 1 | V10-BC15 | ACTCTGGGTCGGTGAGGGTG | 18 |
| 2 | V10-BC17 | ACCCACTGAGTCTCGCGGTG | 19 |
| 3 | V10-BC25 | TCTCTCGCAGGCAGTGGGTG | 20 |
| 4 | V10-BC29 | ACCCTGTGTGACAGGCTGTG | 21 |
| 5 | V3-BC16 | TCACTCGGTCTCTCGCGGTG | 22 |
| 6 | V3-BC19 | ACCCAGTGAGTCAGGGCGTG | 23 |
| 7 | V3-BC9 | TCTCTGTGTCGGTGTCGGTG | 24 |
| 8 | V4-BC2 | AGCGTGTCAGGGTGACCGTG | 25 |
| 9 | V4-BC4 | AGTCTGTCTCTCACAGCGTG | 26 |
| 10 | V4-BC7 | AGAGTGCGTGAGTCTCGCGGTG | 27 |
| 11 | V9-BC18 | ACTGTGGCTCGCTGTCGGTG | 28 |
| 12 | V9-BC19 | ACGGTCTCTCCCAGGCGGTG | 29 |
| 13 | V9-BC21 | TCTCTGCGTGAGTGCCGGTG | 30 |

The barcode region of the GFP-positive cell was PCR-amplified to prepare a library of next-generation sequencer. The library of next-generation sequencer was sequenced in a 600-cycle paired-end mode with MiSeq (Illumina). The obtained sequence data were classified based on the index sequence specific to each sample, and a rate of conversion from GTG into ATG was calculated for each guide RNA used in each experiment (FIG. 5).

As a result, it was confirmed that GTG was converted into ATG with an efficiency of 80% or more in many cases of the barcodes.

It was found that the mutation was repaired in the mutant EGFP by base substitution of GTG to the start codon with a high efficiency, and the EGFP reporter was converted into a wild-type (normal activity was maintained).

### Example 4 Quantitative Evaluation of Specificity to Each of Different Reporter Systems and Efficiency

### <CRISPR Activation, CRISPRa>

It is considered that a barcode-dependent downstream marker gene can be activated at a transcriptional level by using a complex in which a transcription factor was fused with dCas9 (inactive Cas9 mutant). Therefore, the cell population can also be barcoded by the CRISPRa reporter or the guide RNA (gRNA). Therefore, the specificity obtained by the method using the reporter in which ATG was converted into GTG was compared with the specificities obtained by the method using the CRISPRa reporter and the method using the guide RNA.

Specifically, the following three different systems were compared and examined based on the two types of the same barcode sequences, BC4 (AGTCTGTCTCTCACAGCGTG (SEQ ID NO: 31)) and BC6 (AGTCTGGCAGTCACTGGGTG (SEQ ID NO: 32)).

(1) Induction of expression in a cell line having a GTG-EGFP reporter in the genome via a single base substitution by the Cas9 protein-nucleic acid mutation repair enzyme expression vector (CMVp-Sp nCas9-PmCDA1-UGI) and a guide RNA targeting a barcode (GTG-GFP barcode system)

(2) Induction of expression in a cell line having a CRISPRa reporter in the genome (a barcode sequence was cloned into a CRISPRa reporter, and the HEK293Ta cells were infected with the lentivirus, thereby establishing a cell line by selection with puromycin or blasticidin) by a gRNA-dCas9-transcription factor complex (CRISPRa barcode system)

(3) Induction of expression in a cell line having a guide RNA in the genome (a barcode sequence was cloned into a guide RNA for CRISPRa, and the HEK293Ta cells were infected with the lentivirus, thereby establishing a cell line by selection with puromycin or blasticidin) by transfecting cells with the CRISPRa reporter (gRNA barcode system)

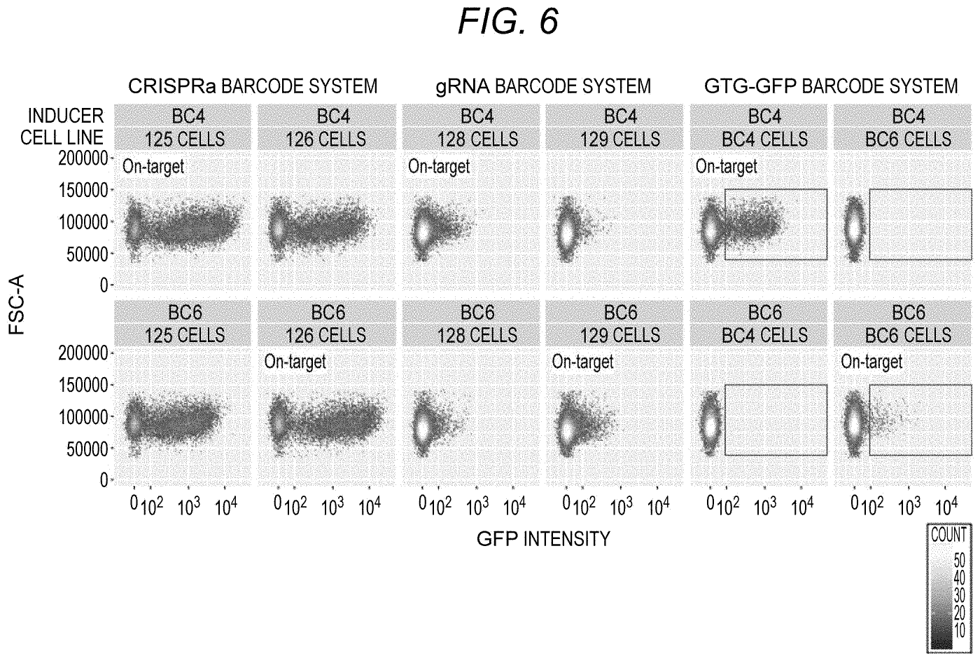

After three days, the cells were collected and a percentage of the GFP-positive cells was analyzed by FACS Verse (manufactured by BD Biosciences). A dot plot showing both two parameters was created by using FSC-A (indicating a cell size) on a vertical axis and FITC (indicating a GFP intensity) on a horizontal axis (FIG. 6). The area on the right side of the value of 10² on the horizontal axis was considered as GFP-positive, and the positive cells were indicated by FITC (GFP intensity).

In the methods of (2) and (3), a large difference in GFP intensities was not observed in each of a combination in which the expression was induced (combination indicated by “On-target” in FIG. 6) and other combinations, whereas in the GTG-GFP barcode system, a significant GFP intensity was observed in the combination in which the expression was induced, which showed the high specificity of the GTG-GFP barcode system (FIG. 6).

In addition, in order to appropriately compare and examine the efficiency of the induction of the expression of GFP and false positives associated therewith by flow cytometry, thresholds of a gate of FITC (GFP) in each of the three systems were continuously changed, and a percentage (% activation) and a false positive (% error) of the GFP-positive cells at each threshold were analyzed and compared.

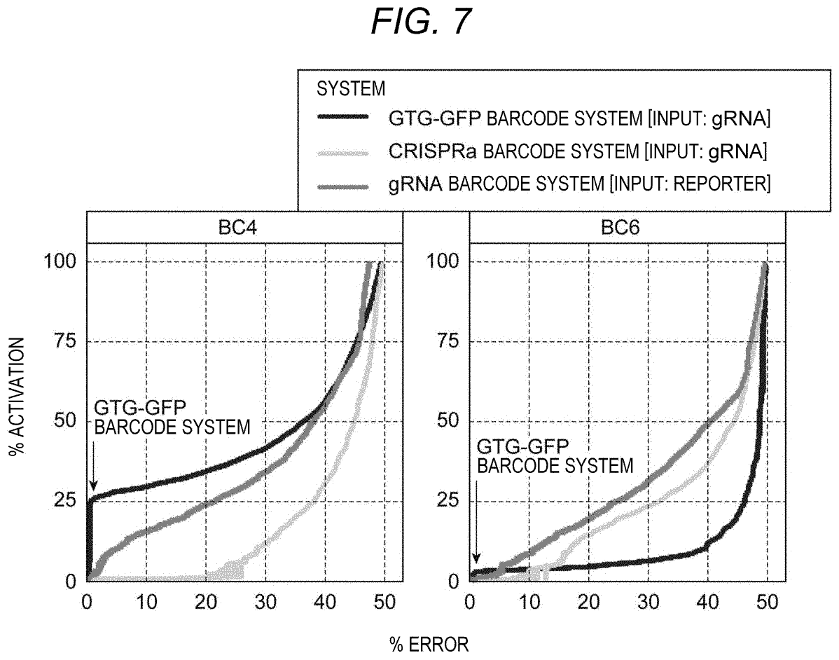

As a result, in the GTG-GFP barcode system, no false positives were detected in a fraction of 3% to 25% of the GFP-positive cells (FIG. 7). On the other hand, about 5% to 20% of the false positives were observed in two transcription-induced systems using CRISPRa.

It was suggested that the reporter expression induction system used in the present invention has excellent performance in terms of both efficiency and false positive.

### Example 5 Demonstration Experiment in Yeast Cells (2)

### <Reporter Abnormal Expression Vector>

A vector consisting of 5′ ADH1 promoter-BsmBI-filler-BsmBI-9^{th}RFP-ADH1 terminator 3′ (SEQ ID NO: 3) was subjected to a restriction enzyme treatment with BsmBI (NEW ENGLAND BioLab, Inc.) (55° C., 1 hour or longer), and a purified product thereof was used as a backbone.

Oligos consisting of sequences of 5′ BsmBI-PAM-barcode-GTG 3′ and 5′ BsmBI-GTG-barcode-PAM 5′ were designed as inserts. The barcode sequence consists of a semi-random barcode represented by (WSNS)₄N. The inserts were amplified by PCR using a primer 1 (5′ ACTGACTGCAGTCTGAGTCTGACAG 3′) (SEQ ID NO: 33) and a primer 2 (5′ CTAGCGTAGAGTGCGTAGCTCTGCT 3′) (SEQ ID NO: 34).

The backbone vector and the insert were mixed with each other in a ratio of 1:10, and the backbone vector and the insert were reacted with each other by a Golden Gate method (at 55° C. for 30 minutes, after repeating a total of 15 cycles at 37° C. for 5 minutes and at 20° C. for 5 minutes). The reacted sample was transformed into E. coli (NEB 5a).

The obtained 100 single colonies were collected from a culture plate, and plasmids were extracted using an extraction kit (NIPPON Genetics Co, Ltd.), thereby obtaining a target DNA barcode pool into which the semi-random DNA barcode was inserted. The sequence of the purified DNA barcode pool was determined by a restriction enzyme treatment and next-generation sequencer.

TABLE 6

| | | SEQ ID |
|---|---|---|
| Plasmid Name | Plasmid Map (Benchling) | NO |
| ADH1p-nCas9- | https://benchling.com/s/seq- | 35 |
| PmCDA1-UGI | ufVDClftYGShzKkDj7oC | |

### <Cas Mutant-Nucleic Acid Mutation Repair Enzyme Expression Vector>

A vector consisting of 5′ ADH1 promotern-nCas9-PmCDA1-UGI-CYC1 terminator 3′ was used as a Cas9 mutant-nucleic acid mutation repair enzyme expression vector (see Table 6, SEQ ID NO: 35).

### <Barcode Recognition Module (Guide RNA) Expression Vector>

A barcode recognition module (guide RNA) expression vector (sgRNA) was constructed as follows.

A vector consisting of 5′ SNR52 promoter-BsmBI-filler-BsmBI-sgRNA scaffold-SUP4 terminator 3′ (SEQ ID NO: 6) was subjected to a restriction enzyme treatment with BsmBI (NEW ENGLAND BioLab, Inc.) (55° C., 1 hour or longer), and a purified product thereof was used as a backbone. A pair of oligos consisting of sequences of 5′ BsmBI-PAM-barcode-GTG 3′ and 5′ BsmBI-GTG-barcode-PAM 5′ were designed as inserts, and a DNA fragment having a BsmBI cut side at a protruding end was obtained by simultaneously performing phosphorylation by T4 polynucleotide kinase (TAKARA BIO INC.) and annealing (in the annealing, a step of lowering the temperature by 1° C. per one cycle in which a reaction was performed for 12 seconds until the temperature reached 25° C. from 95° C. after a reaction at 37° C. for 30 minutes and at 95° C. for 5 minutes was performed, the step being repeated 70 times in total). A barcode recognition sequence of an arbitrary sgRNA was determined from the result of analyzing the sequence of the DNA barcode pool with the next-generation sequencer, by corresponding the barcode recognition sequence (barcode recognition region) to the semi-random DNA barcode sequence represented by (WSNS)₄N. The backbone vector and the insert were mixed with each other in a ratio of 1:10, and the backbone vector and the insert were reacted with each other by a Golden Gate method (at 55° C. for 30 minutes, after repeating a total of 15 cycles at 37° C. for 5 minutes and at 20° C. for 5 minutes). The reacted sample was transformed into E. coli (NEB 5a), and the colonies were cultured and the plasmids were extracted (using an extraction kit from NIPPON Genetics Co, Ltd.), thereby obtaining 12 types of target vectors. A sequence of the purified vector was determined by a Sanger sequencing method. The barcode recognition sequences contained in each of the 12 types of vectors are shown in Table 7.

TABLE 7

| | | | SEQ ID |
|---|---|---|---|
| | Name | Sequence | NO |
| 1 | BC1 | CCACAGCCACCGACCCA | 36 |
| 2 | BC2 | CGCCACTCACAGACGCA | 37 |
| 3 | BC3 | CCAGACTGTGTCTGGCA | 38 |
| 4 | BC4 | ACGCAGCCAGCCTGAGT | 39 |
| 5 | BC5 | AGACACAGACGCAGACA | 40 |
| 6 | BC6 | CCACACCCTGGCTGCCT | 41 |
| 7 | BC7 | GCGCACCGAGTCTGAGT | 42 |
| 8 | BC8 | GCTGAGGGTCACAGCCA | 43 |
| 9 | BC9 | CGGGTCACACCGTCCCA | 44 |
| 10 | BC10 | CGACTCAGACACTCAGT | 45 |
| 11 | BC11 | ACCGTCAGACACTCACA | 46 |
| 12 | 6012 | ACCCACTCTCCGTGAGA | 47 |

<Transformation of Yeast>

A BY4741 strain, which was a standard strain of Saccharomyces cerevisiae, was used as yeast. A commercially available kit (Frozen-EZ Yeast Transformation II™, ZYMO RESEARCH) was used.

First, a DNA barcode pool was transformed into the BY4741 strain. SD-His+Ade was used as an agar medium, and colonies were obtained by culturing at 30° C. for about 48 hours to 72 hours after inoculation. The obtained colonies were collected from the culture plate, competent cells were prepared (Frozen-EZ Yeast Transformation II™, ZYMO RESEARCH) and transformation was performed using each of a Cas9 mutant (nCas9-AID-UGI, SEQ ID NO: 35) and an sgRNA vector (each of 12 types of vectors containing each of barcode recognition sequences of SEQ ID NOs: 36 to 47). SD-His-Leu-Ura+Ade was used as an agar medium, and colonies were obtained by culturing at 30° C. for about 48 hours to 72 hours after inoculation. Barcode sequences of the colonies collected from the culture plate were determined by a next-generation sequencer.

### <Observation of RFP Expression>

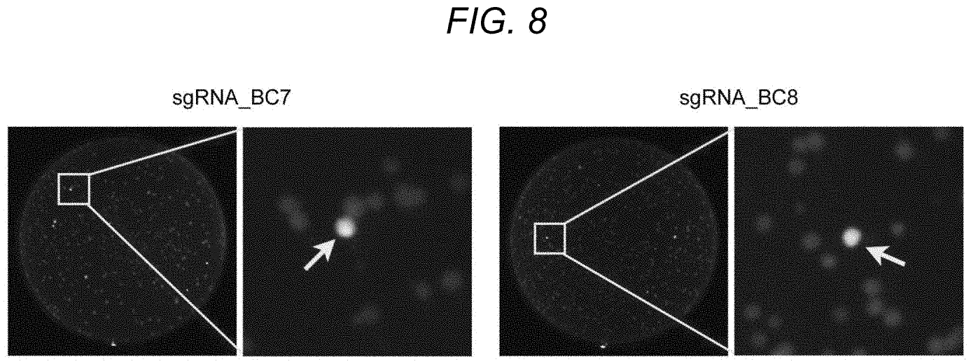

The plate of yeast colonies obtained after transformation of the Cas9 mutant and the sgRNA was irradiated with a blue light built in a gel imaging apparatus (FAS-V, NIPPON Genetics Co, Ltd.), and the colonies emitting red light (RFP expression was predicted) were sampled. FIG. 8 illustrates examples of the sampled colonies, which were predicted to express an RFP. The left shows a result obtained in a case where an sgRNA (sgRNA_BC7) containing a barcode recognition sequence of SEQ ID NO: 42 is used, and the right shows a result obtained in a case where an sgRNA (sgRNA_BC8) containing a barcode recognition sequence of SEQ ID NO: 43 is used.

### <Turbidity Measurement and Fluorescence (RFP) Intensity Measurement>

Turbidity and a fluorescence intensity of each of the yeast colony samples were measured to screen (to observe errors in the sampling of the colonies) RFP expression of the colonies sampled by the blue light irradiation. A microplate reader (Infinite F200 PRO, TECAN Group Ltd.) was used in the measurement. The yeast colonies were cultured and suspended in a selective liquid medium (SD-His-Leu-Ura+Ade), the culture solution was diluted, if necessary, and then, 200 μL of the sample was added to a 96 well-plate (transparent), to measure turbidity. Similarly, 200 μL of the sample was added to a 96 well-plate (black, opaque) to measure a fluorescence intensity. As a result of measuring the turbidity and the fluorescence intensity, it was confirmed that the target colonies were sampled.

### <Determination of Sequences of Sampled Colonies>

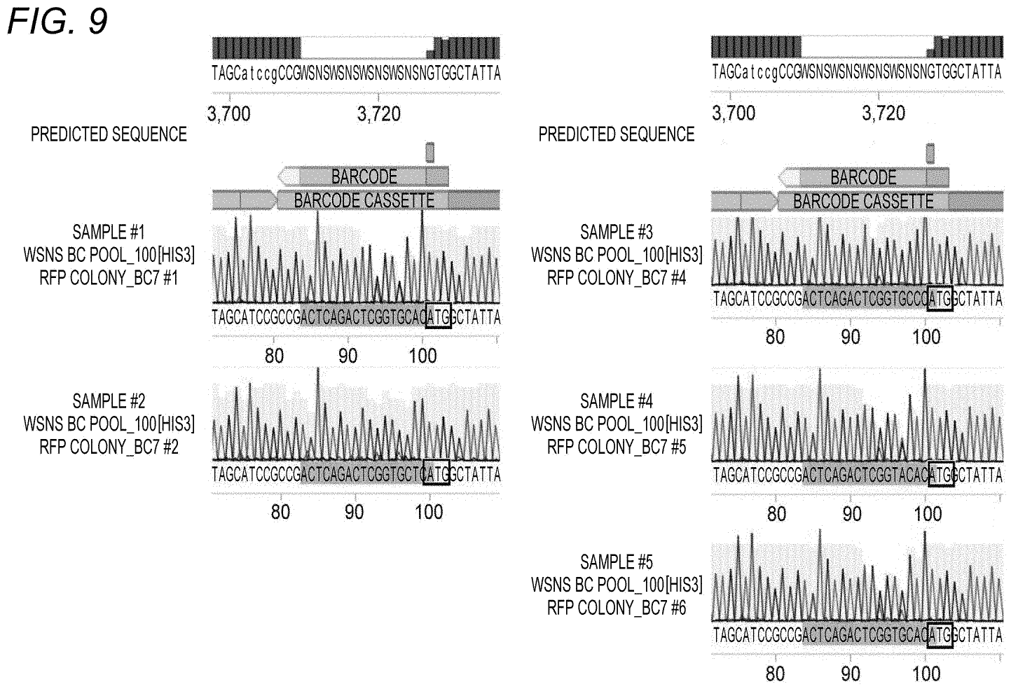

A sequence near the barcode sequence in each of the sampled target colonies was determined by the Sanger sequencing method. As a result, it was confirmed that GTG in the 9^{th}RFP downstream of the barcode sequence was converted into the start codon and the mutation was repaired (FIG. 9).

### Example 6 Verification of Barcode Signal

In order to isolate or identify arbitrary cells from the cell population, it is preferable to observe a single barcode signal in one colony. Therefore, as described below, a barcode signal in a case where the Cas9 protein-nucleic acid mutation repair enzyme expression vector was transformed and then the reporter expression vector was transformed (Method A) was compared with a barcode signal in a case where the reporter expression vector was transformed and then the Cas9 protein-nucleic acid mutation repair enzyme expression vector was transformed (Method B).

### <Reporter Abnormal Expression Vector>

A vector consisting of 5′ ADH1 promoter-BsmBI-filler-BsmBI-9^{th}RFP-ADH1 terminator 3′ (SEQ ID NO: 3) was subjected to a restriction enzyme treatment with BsmBI (NEW ENGLAND BioLab, Inc.) (55° C., 1 hour or longer), and a purified product thereof was used as a backbone.

Oligos consisting of sequences of 5′ BsmBI-PAM-barcode-GTG 3′ and 5′ BsmBI-GTG-barcode-PAM 5′ were designed as inserts. The barcode sequence consists of a semi-random barcode represented by (WSNS)₄N. The inserts were amplified by PCR using a primer 1 (5′ ACTGACTGCAGTCTGAGTCTGACAG 3′) (SEQ ID NO: 33) and a primer 2 (5′ CTAGCGTAGAGTGCGTAGCTCTGCT 3′) (SEQ ID NO: 34).

The backbone vector and the insert were mixed with each other in a ratio of 1:10, and the backbone vector and the insert were reacted with each other by a Golden Gate method (at 55° C. for 30 minutes, after repeating a total of 15 cycles at 37° C. for 5 minutes and at 20° C. for 5 minutes). The reacted sample was transformed into E. coli (NEB 5a).

About 40,000 single colonies obtained were collected from a culture plate, and plasmids were extracted using an extraction kit (NIPPON Genetics Co, Ltd.), thereby obtaining a target DNA barcode pool into which the semi-random DNA barcode was inserted. The sequence of the purified DNA barcode pool was determined by a restriction enzyme treatment and next-generation sequencer.

### <Cas Mutant-Nucleic Acid Mutation Repair Enzyme Expression Vector>

A vector consisting of 5′ ADH1 promotern-nCas9-PmCDA1-UGI-CYC1 terminator 3′ was used as a Cas9 mutant-nucleic acid mutation repair enzyme expression vector (see Table 6, SEQ ID NO: 35).

### <Transformation of Yeast>

A BY4741 strain, which was a standard strain of Saccharomyces cerevisiae, was used as yeast. A commercially available kit (Frozen-EZ Yeast Transformation II™, ZYMO RESEARCH) was used to transform the above-described vector.

### (Experiment Corresponding to the Following Method A)

As a first step, the Cas9 protein-nucleic acid mutation repair enzyme expression vector (Target-AID) was transformed. SD-Leu+Ade was used as an agar medium, and colonies were obtained by culturing at 30° C. for about 48 hours to 72 hours after inoculation.

Competent cells were prepared from the colonies obtained in the first step. A commercially available kit (Frozen-EZ Yeast Transformation II™, ZYMO RESEARCH) was used in the preparation.

As a second step, the reporter expression vector was transformed using the above-described competent cells. SD-His-Leu+Ade was used as an agar medium, and colonies were obtained by culturing at 30° C. for about 48 hours to 72 hours after inoculation.

### (Experiment Corresponding to the Following Method B)

As a first step, the reporter expression vector was transformed. SD-His+Ade was used as an agar medium, and colonies were obtained by culturing at 30° C. for about 48 hours to 72 hours after inoculation.

Competent cells were prepared from the colonies obtained in the first step. A commercially available kit (Frozen-EZ Yeast Transformation II™, ZYMO RESEARCH) was used in the preparation.

As a second step, the Cas9 protein-nucleic acid mutation repair enzyme expression vector (Target-AID) was transformed using the above-described competent cells. SD-His-Leu+Ade was used as an agar medium, and colonies were obtained by culturing at 30° C. for about 48 hours to 72 hours after inoculation.

### <Determination of Sequences of Sampled Colonies>

A sequence near the barcode sequence in each of the sampled single colonies was determined by the Sanger sequencing method. As a result, a sequence in which a plurality of barcode signals was mixed was determined in the sample obtained by transforming the Cas9 protein-nucleic acid mutation repair enzyme expression vector (Target-AID) and then transforming the reporter expression vector (Method A). On the other hand, in the sample obtained by transforming the reporter expression vector and then transforming the Cas9 protein-nucleic acid mutation repair enzyme expression vector (Target-AID) (Method B), a single barcode sequence was determined from each sample, which showed that one colony retained a single plasmid (barcode). In a case where transformation was performed in the order of Method A, the result in which the plurality of barcodes was retained in one colony was not changed even when the DNA concentration in the transformation of the plasmid pool into yeast, the yeast strain to be used, the complexity of the barcode, and the culture time in the liquid medium were changed.

### INDUSTRIAL APPLICABILITY

Furthermore, when the target clone cells are isolated or identified according to the present invention and a unique barcode sequence labeling each cell is specified, it is possible to isolate and analyze unknown cell clones whose marker gene and the like are not obvious from a highly heterogeneous cell population in a marker-free manner. Due to such versatility, the present invention is highly compatible with transcriptome analysis and epigenome analysis of a single cell, which are expected to be further developed and expanded in the future.

1. A method for isolating or identifying target clone cells from a cell population, the method comprising:
a step (i) of preparing a cell population into which a barcode sequence and at least one reporter protein abnormal expression cassette linked to the barcode sequence are introduced;
a step (ii) of introducing a barcode sequence recognition module targeting an arbitrary barcode sequence and a nucleic acid mutation repair enzyme into cells;
a step (iii) of repairing a nucleic acid mutation causing abnormal expression occurring in the at least one reporter protein abnormal expression cassette by expression of a complex of the barcode sequence recognition module and the nucleic acid mutation repair enzyme in a cell containing the target barcode sequence, to induce normal expression of the reporter protein; and
a step (iv) of isolating or identifying target clone cells in which the reporter protein is expressed.

2. The method according to claim 1, wherein the complex converts one or more nucleotides into another one or more nucleotides or deletes the one or more nucleotides, or inserts one or more nucleotides, at a site of the nucleic acid mutation.

3. The method according to claim 1, wherein the nucleic acid mutation is a mutation in a sequence (ATG) encoding methionine which first appears from an N-terminus.

4. The method according to claim 3, wherein the ATG is not included in the barcode sequence.

5. The method according to claim 1, wherein the barcode sequence recognition module is a guide RNA,
the nucleic acid mutation repair enzyme is linked to a Cas protein, and
the guide RNA contains a sequence complementary to at least a part of the barcode sequence.

6. A cell population in which a barcode sequence and at least one reporter protein abnormal expression cassette linked to the barcode sequence are introduced into individual cells.

7. The cell population according to claim 6, wherein a nucleic acid mutation in the at least one reporter protein abnormal expression cassette is a mutation in a sequence (ATG) encoding methionine which first appears from an N-terminus.

8. The cell population according to claim 6, wherein the ATG is not included in the barcode sequence.

9. The cell population according to claim 6, wherein the cell population includes a complex in which a nucleic acid sequence recognition module targeting an arbitrary barcode and a nucleic acid mutation repair enzyme are bound to each other.


SEQUENCE LISTING (47 sequences)

SEQ ID NO: 1 - DNA, 11421 bp, Artificial Sequence
  ADH1p-dCas9
gacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctgggtaccgggcccgaattctctccttctcttaggtggcagagcaggtggagggtcgaccatactagtatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactccatggacaagaagtactccattgggctcgctatcggcacaaacagcgtcggttgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgctatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgggtggaggaggttctggaggtggaggttctgcagagtatgtgcgggccctctttgactttaatgggaatgatgaagaagatcttccctttaagaaaggagacatcctgagaatccgggataagcctgaagagcagtggtggaatgcagaggacagcgaaggaaagagggggatgattcctgtcccttacgtggagaagtattccggagactataaggaccacgacggagactacaaggatcatgatattgattacaaagacgatgacgataagtctagaggctccggataaggatcctaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctcccgggcaaaaaaccccctcaagacccgtttagaggccccaaggggttatgctatgcataccaagcttccgcggtggagctccaattcgccctatagtgagtcgtattacaattcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatcgaccctcgaggagaacttctagtatatccacatacctaatattattgccttattaaaaatggaatcggaacaattacatcaaaatccacattctcttcaaaatcaattgtcctgtacttccttgttcatgtgtgttcaaaaacgttatatttataggataattatactctatttctcaacaagtaattggttgtttggccgagcggtctaaggcgcctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagaggccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatttcaaggatataccattctaatgtctgcccctatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatagtaaaggaatacaggtaagcaaattgatactaatggctcaacgtgataaggaaaaagaattgcactttaacattaatattgacaaggaggagggcaccacacaaaaagttaggtgtaacagaaaatcatgaaactacgattcctaatttgatattggaggattttctctaaaaaaaaaaaaatacaacaaataaaaaacactcaatgacctgaccatttgatggagtttaagtcaataccttcttgaaccatttcccataatggtgaaagttccctcaagaattttactctgtcagaaacggccttacgacgtagtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 2 - DNA, 12386 bp, Artificial Sequence
  ADH1p-dCas9-PmCDA1-UGI
gacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtaccgggcccgaattctctccttctcttaggtggcagagcaggtggagggtcgaccatactagtatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactccatggacaagaagtactccattgggctcgctatcggcacaaacagcgtcggttgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgctatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgggtggaggaggttctggaggtggaggttctgcagagtatgtgcgggccctctttgactttaatgggaatgatgaagaagatcttccctttaagaaaggagacatcctgagaatccgggataagcctgaagagcagtggtggaatgcagaggacagcgaaggaaagagggggatgattcttgtcccttacgtggagaagtattccggagactataaggaccacgacggagactacaaggatcatgatattgattacaaagacgatgacgataagtctaggatgaccgacgctgagtacgtgagaatccatgagaagttggacatctacacgtttaagaaacagtttttcaacaacaaaaaatccgtgtcgcatagatgctacgttctctttgaattaaaacgacggggtgaacgtagagcgtgtttttggggctatgctgtgaataaaccacagagcgggacagaacgtggcattcacgccgaaatctttagcattagaaaagtcgaagaatacctgcgcgacaaccccggacaattcacgataaattggtactcatcctggagtccttgtgcagattgcgctgaaaagatcttagaatggtataaccaggagctgcgggggaacggccacactttgaaaatctgggcttgcaaactctattacgagaaaaatgcgaggaatcaaattgggctgtggaacctcagagataacggggttgggttgaatgtaatggtaagtgaacactaccaatgttgcaggaaaatattcatccaatcgtcgcacaatcaattgaatgagaatagatggcttgagaagactttgaagcgagctgaaaaacgacggagcgagttgtccattatgattcaggtaaaaatactccacaccactaagagtcctgctgtttctagacccaagaagaaaagaaaagtcggtaccatgaccaacctttccgacatcatagagaaggaaacaggcaaacagttggtcatccaagagtcgatactcatgcttcctgaagaagttgaggaggtcattgggaataagccggaaagtgacattctcgtacacactgcgtatgatgagagcaccgatgagaacgtgatgctgctcacgtcagatgccccagagtacaaaccctgggctctggtgattcaggactctaatggagagaacaagatcaagatgctatctagaggctccggataaggatcctaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctcccgggcaaaaaaccccctcaagacccgtttagaggccccaaggggttatgctatgcatactgatataattaaattgaagctctaatttgtgagtttagtatacatgcataccaagcttccgcggtggagctccaattcgccctatagtgagtcgtattacaattcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatcgaccctcgaggagaacttctagtatatccacatacctaatattattgccttattaaaaatggaatcggaacaattacatcaaaatccacattctcttcaaaatcaattgtcctgtacttccttgttcatgtgtgttcaaaaacgttatatttataggataattatactctatttctcaacaagtaattggttgtttggccgagcggtctaaggcgcctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagaggccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatttcaaggatataccattctaatgtctgcccctatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgagttccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatagtaaaggaatacaggtaagcaaattgatactaatggctcaacgtgataaggaaaaagaattgcactttaacattaatattgacaaggaggagggcaccacacaaaaagttaggtgtaacagaaaatcatgaaactacgattcctaatttgatattggaggattttctctaaaaaaaaaaaaatacaacaaataaaaaacactcaatgacctgaccatttgatggagtttaagtcaataccttcttgaaccatttcccataatggtgaaagttccctcaagaattttactctgtcagaaacggccttacgacgtagtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 3 - DNA, 8433 bp, Artificial Sequence
  backbone_ADH1p-filler-RFP [HIS3]
gaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgataagtaaatgcatgtatactaaacccgggctagtacactctatatttttttatgcctcggtaatgattttcatttttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatcatagtcgacagaaattgcatcaacgcatatagcgtatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggcaaattaaagccttcgagcgtcccaaaaccttctcaagcaaggttttcagtataatgttacatgcgtacacgcgtttgtacagaaaaaaaagaaaaatttgaaatataaataacgttcttaatactaacataactattaaaaaaaataaatagggacctagacttcaggttgtctaactccttccttttcggttagagcggatgtgggaggagggcgtgaatgtaagcgtgacataactaattacatgatatcgacaaaggaaaagggctgcagtgctaagcttccaaactaccatcgataggtgcgtgtgaagggaattcactagtatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactgctagcatccggagacggttgtaaatgagcacacaaaatacacatgctaaaatattatattctatgacctttataaaatcaaccaaaatcttctttttaataactttagtatcaataattagaatttttatgttcctttttgcaaacttttaataaaaatgagcaaaataaaaaaacgctagttttagtaactcgcgttgttttcttcacctttaataatagctactccaccacttgttcctaagcggtcagctcctgcttcaatcattttttgagcatcttcaaatgttctaactccaccagctgctttaactaaagcattgtctttaacaactgacttcattagtttaacatcttcaaatgttgcacctgattttgaaaatcctgttgatgttttaacaaattctaatccagcttcaacagctatttcacaagctttcatgatttcttcttttgttaataaacaattttccataatacatttaacaacatgtgatccagctgctttttttacagctttcatgtcttctaaaactaattcataatttttgtcttttaatgcaccaatatttaataccatatcaatttctgttgcaccatctttaattgcttcagaaacttcgaatgcttttgtagctgttgtgcatgcacctagaggaaaacctacaacatttgttattcctacatttgtgccttttaataattctttacaatagcttgttcaatatgaattaacacaaactgttgcaaaatcaaattcaattgcttcatcacataattgtttaatttcagctttcgtagcatcttgttttaataatgtgtgatctatatatttgtttagtttcattttttctcctatatattcatttttaattttaattctttaataatttcgtctactttaactttagcgttttgaacagattcaccaacacctataaaataaatttttagtttaggttcagttccacttgggcgaacagcaaatcatgacttatcttctaaataaaattttagtaagtcttgtcctggcatattatacattccatcgatgtagtcttcaacattaacaactttaagtccagcaatttgagttaagggtgttgctctcaatgatttcattaatggttcaatttttaatttcttttcttctggtttaaaattcaagtttaaagtgaaagtgtaatatgcacccatttctttaaataaatcttctaaatagtctactaatgttttattttgttttttataaaatcaagcagcctctgctattaatatagaagcttgtattccatctttatctctagctgagtcatcaattacatatccataactttcttcataagcaaaaacaaaatttaatccgttatcttcttctttagcaatttctctacccattcatttaaatccagttaaagtttttacaatattaactccatatttttcatgagcgattctatcacccaaatcacttgttacaaaacttgaatatagagccggattttttggaatgctatttaagcgttttagatttgataattttcaatcaattaaaattggtcctgtttgatttccatctaatcttacaaaatgaccatcatgttttattgccattccaaatctgtcagcatctgggtcattcataataataatatctgcatcatgtttaataccatattcaagcggtatttttcatgcaggatcaaattctggatttggatttacaacatttttaaatgtttcatcttcaaatgcatgctcttcaacctcaataacgttatatcctgattcacgtaatatttttggggtaaatttagttcctgttccattaactgcgctaaaaataatttttaaatcttttttagcttcttgctcttttttgtacgtctctgctattattaaagaatttatgagatttaaagttcatatggaaggttcagttaatggtcatgaatttgaaattgaaggtgaaggtgaaggtagaccatatgaaggtactcaaactgctaaattgaaagttactaaaggtggtccattaccatttgcttgggatattttgtcaccacaatttatgtatggttcaaaagcttatgttaaacatccagctgatattccagattatttaaaattgtcatttccagaaggttttaaatgggaaagagttatgaattttgaagatggtggtgttgttactgttactcaagattcatcattacaagatggtgaatttatttataaagttaaattgagaggtactaattttccatcagatggtccagttatgcaaaaaaaaactatgggttgggaagcttcatcagaaagaatgtatccagaagatggtgctttaaaaggtgaaattaaacaaagattgaaattaaaagatggtggtcattatgatgctgaagttaaaactacttataaagctaaaaaaccagttcaattaccaggtgcttataatgttaatattaaattggatattacttcacataatgaagattatactattgttgaacaatatgaaagagctgaaggtagacattcaactggtggtatggatgaattatataaataaaaccggtggtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgcggatcccatctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggc

SEQ ID NO: 4 - DNA, 6576 bp, Artificial Sequence
  reporter_ADH1p-PAM-BC2-9th GTG-RFP [HIS3]
gaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgataagtaaatgcatgtatactaaacccgggctagtacactctatatttttttatgcctcggtaatgattttcatttttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatcatagtcgacagaaattgcatcaacgcatatagcgtatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggcaaattaaagccttcgagcgtcccaaaaccttctcaagcaaggttttcagtataatgttacatgcgtacacgcgtttgtacagaaaaaaaagaaaaatttgaaatataaataacgttcttaatactaacataactattaaaaaaaataaatagggacctagacttcaggttgtctaactccttccttttcggttagagcggatgtgggaggagggcgtgaatgtaagcgtgacataactaattacatgatatcgacaaaggaaaagggctgcagtgctaagcttccaaactaccatcgataggtgcgtgtgaagggaattcactagtatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactgctagcatccgccgagcgtgtcagggtgaccgtggctattattaaagaatttatgagatttaaagttcatatggaaggttcagttaatggtcatgaatttgaaattgaaggtgaaggtgaaggtagaccatatgaaggtactcaaactgctaaattgaaagttactaaaggtggtccattaccatttgcttgggatattttgtcaccacaatttatgtatggttcaaaagcttatgttaaacatccagctgatattccagattatttaaaattgtcatttccagaaggttttaaatgggaaagagttatgaattttgaagatggtggtgttgttactgttactcaagattcatcattacaagatggtgaatttatttataaagttaaattgagaggtactaattttccatcagatggtccagttatgcaaaaaaaaactatgggttgggaagcttcatcagaaagaatgtatccagaagatggtgctttaaaaggtgaaattaaacaaagattgaaattaaaagatggtggtcattatgatgctgaagttaaaactacttataaagctaaaaaaccagttcaattaccaggtgcttataatgttaatattaaattggatattacttcacataatgaagattatactattgttgaacaatatgaaagagctgaaggtagacattcaactggtggtatggatgaattatataaataaaaccggtggtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgcggatcccatctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggc

SEQ ID NO: 5 - DNA, 6576 bp, Artificial Sequence
  reporter_ADH1p-PAM-BC2-9th ATG-RFP [HIS3]
gaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgataagtaaatgcatgtatactaaacccgggctagtacactctatatttttttatgcctcggtaatgattttcatttttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatcatagtcgacagaaattgcatcaacgcatatagcgtatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggcaaattaaagccttcgagcgtcccaaaaccttctcaagcaaggttttcagtataatgttacatgcgtacacgcgtttgtacagaaaaaaaagaaaaatttgaaatataaataacgttcttaatactaacataactattaaaaaaaataaatagggacctagacttcaggttgtctaactccttccttttcggttagagcggatgtgggaggagggcgtgaatgtaagcgtgacataactaattacatgatatcgacaaaggaaaagggctgcagtgctaagcttccaaactaccatcgataggtgcgtgtgaagggaattcactagtatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactgctagcatccgccgagcgtgtcagggtgaccatggctattattaaagaatttatgagatttaaagttcatatggaaggttcagttaatggtcatgaatttgaaattgaaggtgaaggtgaaggtagaccatatgaaggtactcaaactgctaaattgaaagttactaaaggtggtccattaccatttgcttgggatattttgtcaccacaatttatgtatggttcaaaagcttatgttaaacatccagctgatattccagattatttaaaattgtcatttccagaaggttttaaatgggaaagagttatgaattttgaagatggtggtgttgttactgttactcaagattcatcattacaagatggtgaatttatttataaagttaaattgagaggtactaattttccatcagatggtccagttatgcaaaaaaaaactatgggttgggaagcttcatcagaaagaatgtatccagaagatggtgctttaaaaggtgaaattaaacaaagattgaaattaaaagatggtggtcattatgatgctgaagttaaaactacttataaagctaaaaaaccagttcaattaccaggtgcttataatgttaatattaaattggatattacttcacataatgaagattatactattgttgaacaatatgaaagagctgaaggtagacattcaactggtggtatggatgaattatataaataaaaccggtggtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgcggatcccatctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggc

SEQ ID NO: 6 - DNA, 8091 bp, Artificial Sequence
  backbone_SNR52p-filler-sgRNA scaffold
aaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctggagcttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcgagacggttgtaaatgagcacacaaaatacacatgctaaaatattatattctatgacctttataaaatcaaccaaaatcttctttttaataactttagtatcaataattagaatttttatgttcctttttgcaaacttttaataaaaatgagcaaaataaaaaaacgctagttttagtaactcgcgttgttttcttcacctttaataatagctactccaccacttgttcctaagcggtcagctcctgcttcaatcattttttgagcatcttcaaatgttctaactccaccagctgctttaactaaagcattgtctttaacaactgacttcattagtttaacatcttcaaatgttgcacctgattttgaaaatcctgttgatgttttaacaaattctaatccagcttcaacagctatttcacaagctttcatgatttcttcttttgttaataaacaattttccataatacatttaacaacatgtgatccagctgctttttttacagctttcatgtcttctaaaactaattcataatttttgtcttttaatgcaccaatatttaataccatatcaatttctgttgcaccatctttaattgcttcagaaacttcgaatgcttttgtagctgttgtgcatgcacctagaggaaaacctacaacatttgttattcctacatttgtgccttttaataattctttacaatagcttgttcaatatgaattaacacaaactgttgcaaaatcaaattcaattgcttcatcacataattgtttaatttcagctttcgtagcatcttgttttaataatgtgtgatctatatatttgtttagtttcattttttctcctatatattcatttttaattttaattctttaataatttcgtctactttaactttagcgttttgaacagattcaccaacacctataaaataaatttttagtttaggttcagttccacttgggcgaacagcaaatcatgacttatcttctaaataaaattttagtaagtcttgtcctggcatattatacattccatcgatgtagtcttcaacattaacaactttaagtccagcaatttgagttaagggtgttgctctcaatgatttcattaatggttcaatttttaatttcttttcttctggtttaaaattcaagtttaaagtgaaagtgtaatatgcacccatttctttaaataaatcttctaaatagtctactaatgttttattttgttttttataaaatcaagcagcctctgctattaatatagaagcttgtattccatctttatctctagctgagtcatcaattacatatccataactttcttcataagcaaaaacaaaatttaatccgttatcttcttctttagcaatttctctacccattcatttaaatccagttaaagtttttacaatattaactccatatttttcatgagcgattctatcacccaaatcacttgttacaaaacttgaatatagagccggattttttggaatgctatttaagcgttttagatttgataattttcaatcaattaaaattggtcctgtttgatttccatctaatcttacaaaatgaccatcatgttttattgccattccaaatctgtcagcatctgggtcattcataataataatatctgcatcatgtttaataccatattcaagcggtatttttcatgcaggatcaaattctggatttggatttacaacatttttaaatgtttcatcttcaaatgcatgctcttcaacctcaataacgttatatcctgattcacgtaatatttttggggtaaatttagttcctgttccattaactgcgctaaaaataatttttaaatcttttttagcttcttgctcttttttgtacgtctctgttttagagctagaaatagcaagttaaaataaggctagtccgttatcaacttgaaaaagtggcaccgagtcggtggtgctttttttgttttttatgtcttcgagtcatgtaattagttatgtcacgcttacgttcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgcaccgcacggtcaccctgaacattatactgaaaaccttgcttgagaaggttttgggacgctcgaaggctttaatttgcggccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcatctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaattgaaaagctgtggtatggtgcactctcagtacaatctgctctgatgccgcatagttaagccagccccgacacccgccaacacccgctgacgcgccctgacgggcttgtctgctcccggcatccgcttacagacaagctgtgac

SEQ ID NO: 7 - DNA, 6231 bp, Artificial Sequence
  sgRNA_SNR52p-target BC2
aaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctggagcttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatccacggtcaccctgacacgctgttttagagctagaaatagcaagttaaaataaggctagtccgttatcaacttgaaaaagtggcaccgagtcggtggtgctttttttgttttttatgtcttcgagtcatgtaattagttatgtcacgcttacgttcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgcaccgcacggtcaccctgaacattatactgaaaaccttgcttgagaaggttttgggacgctcgaaggctttaatttgcggccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcatctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaattgaaaagctgtggtatggtgcactctcagtacaatctgctctgatgccgcatagttaagccagccccgacacccgccaacacccgctgacgcgccctgacgggcttgtctgctcccggcatccgcttacagacaagctgtgac

SEQ ID NO: 8 - DNA, 6270 bp, Artificial Sequence
  sgRNA_SNR52p-scrambled
gacgaaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctggagcttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcctgaaaaaggaaggagttgagttttagagctagaaatagcaagttaaaataaggctagtccgttatcaacttgaaaaagtggcaccgagtcggtggtgctttttttgttttttatgtcttcgagtcatgtaattagttatgtcacgcttacgttcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaattgaaaagctgtggtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 9 - DNA, 17 bp, Artificial Sequence
  Barcode Sequence
agcgtgtcagggtgacc

SEQ ID NO: 10 - DNA, 20 bp, Artificial Sequence
  Spacer Sequence
cacggtcaccctgacacgct

SEQ ID NO: 11 - DNA, 5822 bp, Artificial Sequence
  pMD2.G
ggatcccctgagggggcccccatgggctagaggatccggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattctgacactatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaactcaaatcctgcacaacagattcttcatgtttggaccaaatcaacttgtgataccatgctcaaagaggcctcaattatatttgagtttttaatttttatgaaaaaaaaaaaaaaaaacggaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgt

SEQ ID NO: 12 - DNA, 10668 bp, Artificial Sequence
  psPAX2
agccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgggccgcgttgacgcgcacggcaagaggcgaggggcggcgactggtgagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaattctgcaacaactgctgtttatccatttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagtaagtagtacatgtaatgcaacctataatagtagcaatagtagcattagtagtagcaataataatagcaatagttgtgtggtccatagtaatcatagaatataggaaaatggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtgctgttagcttgctcaatgccacagccatagcagtagctgaggggacagatagggttatagaagtagtacaaggagcttgtagagctattcgccacatacctagaagaataagacagggcttggaaaggattttgctataagctcgaaacaaccggtacctctagaactatagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgc

SEQ ID NO: 13 - DNA, 8367 bp, Artificial Sequence
  pLV-CS-110 (lenti-T002-GTG-EGFP)
gcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctactagaggatctatttccggtgaattctaatacgactcactatagggagacccaagctggctagcccgaactataacatcatttcgtggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggatccaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagttaacgccgccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctagacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgttttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaactttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatc

SEQ ID NO: 14 - DNA, 20 bp, Artificial Sequence
  T002 Barcode Sequence
aactataacatcatttcgtg

SEQ ID NO: 15 - DNA, 8322 bp, Artificial Sequence
  pLV-CS-076 (lentiGuide-T002)
ccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccagagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcacgaaatgatgttatagttgttttagagctagaaatagcaagttaaaataaggctagtccgttatcaacttgaaaaagtggcaccgagtcggtgcttttttaagcttggcgtaactagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggcgccggctcgagggggcccgggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgccgtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcaga

SEQ ID NO: 16 - DNA, 8322 bp, Artificial Sequence
  pLV-CS-077 (lentiGuide-Scramble1)
ccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccagagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgaaaaaggaaggagttgagttttagagctagaaatagcaagttaaaataaggctagtccgttatcaacttgaaaaagtggcaccgagtcggtgcttttttaagcttggcgtaactagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggcgccggctcgagggggcccgggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgccgtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcaga

SEQ ID NO: 17 - DNA, 10419 bp, Artificial Sequence
  pcDNA3.1_pCMV-nCas-PmCDA1-ugi pH1-gRNA(HPRT)
ggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgaggaccacagatccccccgagatgtcatgaaagagagttttagagctagaaatagcaagttaaaataaggctagtccgttatcaacttgaaaaagtggcaccgagtcggtgctttttttacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgaaatggcaccgaagaagaagcgtaaagtcggaatccacggagttcctgcggcaatggacaagaagtactccattgggctcgctatcggcacaaacagcgtcggttgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgggtggaggaggtaccggcggtggaggctcagcagaatacgtacgagctctgtttgacttcaatgggaatgacgaggaggatctcccctttaagaagggcgatattctccgcatcagagataagcccgaagaacaatggtggaatgccgaggatagcgaagggaaaaggggcatgattctggtgccatatgtggagaaatattccggtgactacaaagaccatgatggggattacaaagaccacgacatcgactacaaagacgacgacgataaatcagggatgacagacgccgagtacgtgcgcattcatgagaaactggatatttacaccttcaagaagcagttcttcaacaacaagaaatctgtgtcacaccgctgctacgtgctgtttgagttgaagcgaaggggcgaaagaagggcttgcttttggggctatgccgtcaacaagccccaaagtggcaccgagagaggaatacacgctgagatattcagtatccgaaaggtggaagagtatcttcgggataatcctgggcagtttacgatcaactggtattccagctggagtccttgcgctgattgtgccgagaaaattctggaatggtataatcaggaacttcggggaaacgggcacacattgaaaatctgggcctgcaagctgtactacgagaagaatgcccggaaccagataggactctggaatctgagggacaatggtgtaggcctgaacgtgatggtttccgagcactatcagtgttgtcggaagattttcatccaaagctctcataaccagctcaatgaaaaccgctggttggagaaaacactgaaacgtgcggagaagtggagatccgagctgagcatcatgatccaggtcaagattctgcataccactaagtctccagccgttggtcccaagaagaaaagaaaagtcggtaccatgaccaacctttccgacatcatagagaaggaaacaggcaaacagttggtcatccaagagtcgatactcatgcttcctgaagaagttgaggaggtcattgggaataagccggaaagtgacattctcgtacacactgcgtatgatgagagcaccgatgagaacgtgatgctgctcacgtcagatgccccagagtacaaaccctgggctctggtgattcaggactctaatggagagaacaagatcaagatgctagaaggcaggggaagccttctgacttgtggggatgtggaagaaaaccctggtccatctagaatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttagc

SEQ ID NO: 18 - DNA, 20 bp, Artificial Sequence
  V10-BC15
actctgggtcggtgagggtg

SEQ ID NO: 19 - DNA, 20 bp, Artificial Sequence
  V10-BC17
acccactgagtctcgcggtg

SEQ ID NO: 20 - DNA, 20 bp, Artificial Sequence
  V10-BC2
tctctcgcaggcagtgggtg

SEQ ID NO: 21 - DNA, 20 bp, Artificial Sequence
  V10-BC29
accctgtgtgacaggctgtg

SEQ ID NO: 22 - DNA, 20 bp, Artificial Sequence
  V3-BC16
tcactcggtctctcgcggtg

SEQ ID NO: 23 - DNA, 20 bp, Artificial Sequence
  V3-BC19
acccagtgagtcagggcgtg

SEQ ID NO: 24 - DNA, 20 bp, Artificial Sequence
  V3-BC9
tctctgtgtcggtgtcggtg

SEQ ID NO: 25 - DNA, 20 bp, Artificial Sequence
  V4-BC2
agcgtgtcagggtgaccgtg

SEQ ID NO: 26 - DNA, 20 bp, Artificial Sequence
  V4-BC4
agtctgtctctcacagcgtg

SEQ ID NO: 27 - DNA, 22 bp, Artificial Sequence
  V4-BC7
agagtgcgtgagtctcgcggtg

SEQ ID NO: 28 - DNA, 20 bp, Artificial Sequence
  V9-BC18
actgtggctcgctgtcggtg

SEQ ID NO: 29 - DNA, 20 bp, Artificial Sequence
  V9-BC19
acggtctctcccaggcggtg

SEQ ID NO: 30 - DNA, 20 bp, Artificial Sequence
  V9-BC21
tctctgcgtgagtgccggtg

SEQ ID NO: 31 - DNA, 20 bp, Artificial Sequence
  BC4
agtctgtctctcacagcgtg

SEQ ID NO: 32 - DNA, 20 bp, Artificial Sequence
  BC6
agtctggcagtcactgggtg

SEQ ID NO: 33 - DNA, 25 bp, Artificial Sequence
  Primer 1
actgactgcagtctgagtctgacag

SEQ ID NO: 34 - DNA, 25 bp, Artificial Sequence
  Primer 2
ctagcgtagagtgcgtagctctgct

SEQ ID NO: 35 - DNA, 12386 bp, Artificial Sequence
  ADH1p-nCas9-PmCDA1-UGI
gacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtaccgggcccgaattctctccttctcttaggtggcagagcaggtggagggtcgaccatactagtatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactccatggacaagaagtactccattgggctcgctatcggcacaaacagcgtcggttgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgggtggaggaggttctggaggtggaggttctgcagagtatgtgcgggccctctttgactttaatgggaatgatgaagaagatcttccctttaagaaaggagacatcctgagaatccgggataagcctgaagagcagtggtggaatgcagaggacagcgaaggaaagagggggatgattcttgtcccttacgtggagaagtattccggagactataaggaccacgacggagactacaaggatcatgatattgattacaaagacgatgacgataagtctaggatgaccgacgctgagtacgtgagaatccatgagaagttggacatctacacgtttaagaaacagtttttcaacaacaaaaaatccgtgtcgcatagatgctacgttctctttgaattaaaacgacggggtgaacgtagagcgtgtttttggggctatgctgtgaataaaccacagagcgggacagaacgtggcattcacgccgaaatctttagcattagaaaagtcgaagaatacctgcgcgacaaccccggacaattcacgataaattggtactcatcctggagtccttgtgcagattgcgctgaaaagatcttagaatggtataaccaggagctgcgggggaacggccacactttgaaaatctgggcttgcaaactctattacgagaaaaatgcgaggaatcaaattgggctgtggaacctcagagataacggggttgggttgaatgtaatggtaagtgaacactaccaatgttgcaggaaaatattcatccaatcgtcgcacaatcaattgaatgagaatagatggcttgagaagactttgaagcgagctgaaaaacgacggagcgagttgtccattatgattcaggtaaaaatactccacaccactaagagtcctgctgtttctagacccaagaagaaaagaaaagtcggtaccatgaccaacctttccgacatcatagagaaggaaacaggcaaacagttggtcatccaagagtcgatactcatgcttcctgaagaagttgaggaggtcattgggaataagccggaaagtgacattctcgtacacactgcgtatgatgagagcaccgatgagaacgtgatgctgctcacgtcagatgccccagagtacaaaccctgggctctggtgattcaggactctaatggagagaacaagatcaagatgctatctagaggctccggataaggatcctaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctcccgggcaaaaaaccccctcaagacccgtttagaggccccaaggggttatgctatgcatactgatataattaaattgaagctctaatttgtgagtttagtatacatgcataccaagcttccgcggtggagctccaattcgccctatagtgagtcgtattacaattcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatcgaccctcgaggagaacttctagtatatccacatacctaatattattgccttattaaaaatggaatcggaacaattacatcaaaatccacattctcttcaaaatcaattgtcctgtacttccttgttcatgtgtgttcaaaaacgttatatttataggataattatactctatttctcaacaagtaattggttgtttggccgagcggtctaaggcgcctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagaggccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatttcaaggatataccattctaatgtctgcccctatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgagttccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatagtaaaggaatacaggtaagcaaattgatactaatggctcaacgtgataaggaaaaagaattgcactttaacattaatattgacaaggaggagggcaccacacaaaaagttaggtgtaacagaaaatcatgaaactacgattcctaatttgatattggaggattttctctaaaaaaaaaaaaatacaacaaataaaaaacactcaatgacctgaccatttgatggagtttaagtcaataccttcttgaaccatttcccataatggtgaaagttccctcaagaattttactctgtcagaaacggccttacgacgtagtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 36 - DNA, 17 bp, Artificial Sequence
  BC1
ccacagccaccgaccca

SEQ ID NO: 37 - DNA, 17 bp, Artificial Sequence
  BC2
cgccactcacagacgca

SEQ ID NO: 38 - DNA, 17 bp, Artificial Sequence
  BC3
ccagactgtgtctggca

SEQ ID NO: 39 - DNA, 17 bp, Artificial Sequence
  BC4
acgcagccagcctgagt

SEQ ID NO: 40 - DNA, 17 bp, Artificial Sequence
  BC5
agacacagacgcagaca

SEQ ID NO: 41 - DNA, 17 bp, Artificial Sequence
  BC6
ccacaccctggctgcct

SEQ ID NO: 42 - DNA, 17 bp, Artificial Sequence
  BC7
gcgcaccgagtctgagt

SEQ ID NO: 43 - DNA, 17 bp, Artificial Sequence
  BC8
gctgagggtcacagcca

SEQ ID NO: 44 - DNA, 17 bp, Artificial Sequence
  BC9
cgggtcacaccgtccca

SEQ ID NO: 45 - DNA, 17 bp, Artificial Sequence
  BC10
cgactcagacactcagt

SEQ ID NO: 46 - DNA, 17 bp, Artificial Sequence
  BC11
accgtcagacactcaca

SEQ ID NO: 47 - DNA, 17 bp, Artificial Sequence
  BC12
acccactctccgtgaga